(19) United States
(12) Patent Application Publication
(10) Pub. No.: US 2009/0075270 A1
(43) Pub. Date: Mar. 19, 2009

(54) Products and Methods Relating to the Use of the Endoribonuclease Kid/PemK

(75) Inventors: Guillermo de la Cueva-Mendez, Cambridge (GB); Belen Pimentel de Francisco, Cambridge (GB)

Correspondence Address: MCANDREWS HELD & MALLOY, LTD, 500 WEST MADISON STREET, SUITE 3400, CHICAGO, IL, 60661, US

(73) Assignee: Medical Research Council, London (GB)

(21) Appl. No.: 12/066,715

(22) PCT Filed: Sep. 13, 2006
(86) PCT No.: PCT/GB06/03385

§ 371 Date: Sep. 8, 2008

(30) Foreign Application Priority Data
Sep. 14, 2005 (GB) 0518777.8

(51) Int. Cl. C12Q 1/68; C07H 21/02; C12N 5/00; C12P 19/34; C12N 15/66; C12N 15/85

(52) U.S. Cl. 435/6; 536/23.1; 435/320.1; 435/377; 435/91.4; 435/91.1

(57) ABSTRACT

The invention relates to a method for engineering a nucleic acid for expression in the presence of Kid/PemK endoribonuclease comprising (i) screening the nucleotide sequence of the nucleic acid for the sequence UUACU or TTACT (ii) mutating said sequence such that there are no longer any occurrences of UUACU or TTACT. The invention also relates to a method of making a ribonucleic acid resistant to Kid/PemK endoribonuclease, said method comprising (a) providing a nucleic acid; (b) screening the nucleic acid for the nucleotide sequence UUACU or TTACT; (c) mutating said sequence such that there are no longer any occurrences of UUACU or TTACT; wherein when the nucleic acid of (a) is a deoxyribonucleic acid, said method further comprises (d) transcribing said deoxyribonucleic acid to produce ribonucleic acid. The invention also relates to vectors and uses of purified or recombinant Kid/PemK endoribonucleases.

### FIELD OF THE INVENTION

The invention is in the field of molecular biology, in particular in the field of endoribonuclease action on RNA. The invention relates to methods for evading the action of Kid (PemK) endoribonuclease, to methods for manipulating nucleic acid expression, and to nucleic acids which have been modified in order to resist Kid/PemK action.

### BACKGROUND TO THE INVENTION

The copy number of many extrachromosomal elements is tightly regulated. Examples range from bacterial plasmids and phages to human viruses such as Epstein Barr virus (EBV), herpes simplex virus (HSV), or human papilloma virus (HPV). In spite of major efforts on a wide range of biological systems, many important aspects of copy number control have remained elusive.

Kid and Kis are members of a larger family of toxin-antitoxin pairs. They are encoded by the parD locus of E. coli plasmid R1 and are conserved in a closely related plasmid, R100, and also have chromosomal homologues in E. coli. These chromosomal homologues are functionally and structurally related to Kid and Kis. They function as stress response elements and their toxins are cytostatic and reversible, which is essential for their biological function. Interestingly, although parD was described as a post-segregational killing system, it was reported later that Kid only exerts a cytostatic effect. Recent studies have shed light on the mode of action of these toxins—they are endoribonucleases that cleave cellular RNAs and inhibit protein synthesis. However, some discrepancies exist regarding the nature of the RNAs and the particular sequences that they cleave.

The control of replication of plasmid R1 and the parD system have been extensively studied, but understanding is still limited. Under normal circumstances, CopB contributes very little to the control of R1 copy number, so it is unclear why CopB has been retained by R1. It has been suggested that copB could have been maintained by R1 to act as a rescue system when copy number of the plasmid is very low, but how the rescue system works remains to be established. Similarly, the contribution of parD to plasmid stability is only evident in a replication defective mutant of plasmid R1, but the reasons for this are not fully understood.

Zhang et al (2004 Journal of Biological Chemistry Volume 279 pages 20678-20684) disclose the interference in mRNA function by sequence specific endoribonuclease PemK. PemK and PemI are a toxin/anti-toxin pair found on plasmid R100. It has been shown that PemK and PemI are identical to Kid and Kis, a toxin/anti-toxin pair found on plasmid R1. Zhang et al mention the previous discovery that Kid (PemK) and Kis (PemI) not only function in bacteria but also function efficiently in a wide range of eukaryotes (de la Cueva-Mendez et al 2003 EMBO vol 22 pp 246-251). It has been previously shown that Kid inhibits cell proliferation in yeast, Xeiiopus laevis, and human cells, and that the inhibition was released by Kis. Zhang et al focus on the sequence specificity of the PemK endoribonuclease. They disclose that the primary cleavages occur at the 5′ or 3′ side of the A residue in the UAH sequence (where H is C, A or U). However, Zhang et al also discussed the relevance of the UA dinucleotide, and also report cleavage at a UGC sequence. Overall, their analysis appears to show that UAC is the most common cleavage site, appearing at 11 of the 18 cleavage sites determined in their study. Although some basic rules of sequence specificity can be derived from Zhang et al, there is no disclosure of a fully defined recognition site for Kid (PemK). Zhang et al also mentioned previous reports that Kid can trigger apoptosis in human cancer cells, which can be inhibited by Kis (de la Cueva-Mendez et al 2003 EMBO vol 22 pp 246-251). Furthermore, Zhang et al suggest that this mRNA interfering system could be used for gene therapy for human diseases.

Muñoz-Gómez et al (2005 J Bacteriology Volume 187 pages 3151-3157) disclose RNAse/anti-RNAse activities of the bacterial parD toxin-antitoxin system. The parD systems encodes the Kid and Kis toxin/antitoxin pair. Muñoz-Gómez et al show that Kid cleaves RNA and inhibits protein synthesis in rabbit reticulocyte lysates. The mechanism of action of Kid and a partially inactive Kid mutant is studied. FIG. 2c presents various observed cleavage sites of Kid. The consensus site which is derived is a UA dinucleotide with an A/C third position. The conclusions drawn about the probable cleavage site for Kid agree with the conclusions of Zhang et al, but cleavage at related sites such as UAG is also reported, together with a preference for UAA or UAC sites.

Thus, from the prior art, the specificities of the Kid endoribonuclease appear to be ill defined. Whilst there is some agreement, there is also some divergence. Furthermore, a common teaching running through the prior art is that Kid has a very minimal recognition sequence, focused mainly on the UA dinucleotide with a variable third position. Thus, it would appear that the number of Kid recognition sites present in RNAs transcribed in cells would be high. With only a 2/3 nucleotide recognition sequence, the expectation is that Kid would cleave virtually all naturally occurring mRNAs.

Suzuki et al (2005 Molecular Cell Volume 18 pages 253-261) disclose single protein production in living cells facilitated by an mRNA interferase called a MazF. MazF targets RNAse and selectively degrades those having an ACA sequence. Thus, MazF is a bacterial toxin which is a single stranded RNA endoribonuclease specific for the ACA sequence. Suzuki et al exploit the unique cleavage properties of MazF to design their single protein production system. Essentially, a gene is engineered to express an ACA-less mRNA. MazF expression is then induced. MazF protein selectively degrades all RNAse having the ACA sequence. Since the expression sequence has been engineered to be ACA less, then this RNA is not degraded. Therefore, protein production continues from this ACA-less mRNA and the protein of interest comes to comprise a very high proportion of the total cellular protein. Suzuki et al explain how silent mutations can be made when ACA occurs in each of the three possible reading frames, although compensating changes often have to be made in neighbouring sites. Suzuki et al apply their technique to eukaryotic proteins such as yeast proteins. In summary, Suzuki et al applied the distinctive enzymatic properties of the bacterial toxin MazF to develop a protein expression system which yields high signal to noise ratios via the selective activity of MazF. This was shown to be better than existing PET based systems or pCOLD based systems. However, ACA is a very common triplet found in a variety of RNAs. Furthermore, because of its short sequence, options for mutating it are quite limited. Since it is only three nucleotides in length, frequent mutation events are necessary in order to engineer an mRNA to be resistant to the action of MazF. Furthermore, when the ACA sequence is found in the +3 reading frame, it can cause problems in neighbouring codons and it can be quite awkward to maintain the correct coding sequence whilst removing the ACA by mutation. Nowhere in Suzuki et al is it taught or suggested to use Kid endoribonuclease in a SPP system. In fact, on page 253 of Suzuki et al it is commented that PemK (PemK=Kid) has been shown to be a sequence specific endoribonuclease that possesses broader coverage specificity than MazF. Thus, this clearly teaches the skilled reader that Kid would be even more difficult to use than MazF since it attacks a wider range of RNA sequences.

The present invention seeks to overcome problems associated with the prior art.

### SUMMARY OF THE INVENTION

The present invention is based on the surprising finding that Kid endoribonuclease is more specific than was previously thought. The Kid recognition site is 5′-UUACU-3′ (abbreviated to UUACU). This site has been defined where it has not been previously elucidated. In the prior art, it has been regarded as a dinucleotide (UA) with some disagreement about the relevance of the third nucleotide following the dinucleotide UA. However, by studying the processes in vivo in the context of plasmid R1 stability, the present inventors have developed a better understanding of the biological specificity of Kid. The present invention is based on these findings.

The invention finds broad application as is explained below. However, one important application of the invention is in single protein production systems. The prior art systems have been based on the specific sequence recognition properties of the MazF interferase. Indeed, the prior art specifically teaches away from the use of Kid because it was thought to have a broader sequence specificity than MazF which would lead to it digesting a greater number of mRNA sites than MazF. However, it has been surprisingly found by the present inventors that Kid in fact has a very specific 5 nucleotide recognition sequence of UUACU. This offers numerous advantages over prior art such as MazF protein production systems. For example, by working the prior art it is necessary to make a very large number of mutations in order to engineer the gene to be resistant to the action of MazF since its recognition site is only 3 nucleotides long. This is extremely onerous. Furthermore, based on the prior art understanding of the Kid recognition site, potentially even more mutations would need to be made in a RNA in order to make it resistant to the action of Kid. However, based on the findings disclosed herein, RNAs can be advantageously made resistant to endoribonuclease action using a far lower degree of mutation than was previously thought necessary. This leads to significant labour and costs savings, as well as simplifying the system and providing greater flexibility in mutating the recognition sites without altering the encoded amino acid sequences.

Therefore, it can be appreciated that the invention provides numerous products and methods based on a deeper understanding of the action and behaviour of Kid endoribonuclease.

Thus, in a first aspect the invention provides a method for engineering a nucleic acid for expression in the presence of Kid/PemK endoribonuclease comprising (i) screening the nucleotide sequence of the nucleic acid for the sequence UUACU or TTACT; (ii) mutating said sequence such that there are no longer any occurrences of UUACU or TTACT.

In the present invention, screening has its normal meaning and refers to scanning, reviewing, reading, analysing, examining or otherwise interrogating the nucleotide sequence of the nucleic acid of interest. If that sequence is not known, then preferably the screening step comprises two parts, namely determination of the sequence followed by screening of same. Determination of the sequence of a nucleic acid such as a cloned nucleic acid is routine for a person skilled in the art. A service company could be engaged to perform the sequence determination. Alternatively, numerous manufacturers' kits are commercially available containing all of the reagents necessary. Preferred techniques are highlighted in the examples section below.

In one embodiment, screening/mutating refers to the whole nucleic acid construct. Preferably screening/mutating is within the expressed region. Preferably screening/mutating is within the transcribed region. For constructs encoding polypeptides, preferably screening/mutating is within the translated region; preferably within exons of genes with introns; preferably within the full open reading frame.

In the present invention, expression means production of RNA, such as biologically active RNAs, or RNAs encoding polypeptides for translation. Many nucleic acid constructs are used for polypeptide production through the intermediate of RNA, thus preferably ‘expression’ includes production of polypeptide by translation of said RNA. Preferably said RNA is stable in the presence of Kid/PemK. ‘Stable’ means that it will not be degraded/digested/cleaved by Kid/PemK—whether it is fully stable in the particular environment in which it is produced is a matter for the operator and may involve a consideration of other endoribonucleases (and/or exoribonucleases) which may be present. In the context of the current specification, ‘stable’ refers principally to stability with respect to Kid/PemK endoribonuclease. Preferably stable means is not cleaved by Kid/PemK.

The nucleic acid may be any suitable nucleic acid such as a recombinant nucleic acid construct such as a gene construct. Preferably the nucleic acid comprises a promoter operably linked to an open reading frame (ORF) of a polypeptide of interest. Further elements such as enhancers, stop boxes, further promoters, processing signals and the like are well known to the person skilled in the art and their selection and/or inclusion is not a core part of the present invention except where stated, except to the degree that they may confer sensitivity to Kid/PemK for example by possession of TTACT/UUACU recognition sequence(s); according to the present invention these are advantageously removed by mutation from each element of the nucleic acid/construct, or at least those elements which will exist as RNA during the life of the nucleic acid/construct, preferably those elements which will exist as ssRNA during the life of the nucleic acid/construct.

Preferably each occurrence of UUACU or TTACT is mutated by replacing the first or last U/T of each occurrence. Preferably said first or last U or T is mutated as a silent mutation. Preferably the last U/T is mutated wherever possible, and preferably as a silent mutation.

When the nucleic acid is for use in directing production of a polypeptide, preferably mutations take account of the codon in which they are made. Preferably conservative mutations are made. More preferably, silent mutations are made. As is known to a person skilled in the art, a silent mutation is one which is made with respect to the amino acid specified by the codon being mutated, and is chosen in such a way as to not change the amino acid specified. It is an advantage of the present invention that each of the occurrences of the first or last U or T in the Kid/PemK recognition site can be mutated to remove the Kid/PemK recognition site but without changing the amino acids encoded across the unmutated UUACU site. A person skilled in the art can easily choose the appropriate mutations on the basis of this teaching. Nevertheless, in outline, mutations are preferably chosen as follows. Firstly, the site to be mutated is first identified by screening the sequence as discussed above. The site could be in any of the three possible reading frames (ie. UUA, CUX; XUU, ACU; XXU, UAC). With reference to the coding sequence of the polypeptide of interest, the correct open reading frame is determined for that occurrence of UUACU. With reference to the genetic code, a silent mutation is then chosen which destroys the UUACU recognition site, but which does not change the amino acid encoded. Balancing mutations may be made to restore a codon eg. it may be desired to change two nucleotides to maintain the codon whilst removing the UUACU sequence. It is preferred to make the fewest possible number of mutations. It is preferred to only mutate the first or last U/T of the UUACU sequence. Advantageously the present invention allows all occurrences of UUACU to be removed by mutating only the first and/or last U/T of the UUACU sequence. It is a further advantage of the present invention that the site can be removed by silent mutation by mutating only the first and/or last U/T of the UUACU sequence. Preferably only the first or the last U/T of the sequence is mutated, ie. preferably only one nucleotide is changed in the UUACU site. Preferably the mutation is a silent mutation. In this embodiment, the choice whether to mutate the 5′ U/T or the 3′ U/T may be made by the operator. Preferably the one which allows silent mutation is chosen. If both allow silent mutation, or the replacement nucleotide may be one of several each being a silent mutation, then in general codon preference of the destination organism (ie. the organism in which the nucleic acid will be used to direct polypeptide expression) is taken into account to assist in choosing the most efficient replacement nucleotide for the intended application. Preferably the most commonly used codon for that amino acid residue in the destination organism is selected, or the most highly used codon possible within the constraints of making the mutation (for example if the most commonly used codon would not destroy the UUACU site). Preferably no neighbouring nucleotides (ie. those not comprised by the UUACU site) are mutated.

Thus, advantageously every single combination of amino acids arising from reading TTACT in any of the three possible reading frames can be maintained by mutation only of the first or last T/U of the TTACT (UUACU) sequence (ie. by changing only either position one or position five of that sequence). This new coding sequence will advantageously be resistant to Kid cleavage but will maintain the primary sequence in the protein.

If it is desired to mutate the core ‘TAC’ sequence of the TTACT site, preferably TAC is mutated to TAT. This TAT is regarded as a cleavage site in the prior art, but is surprisingly shown herein to be resistant to Kid cleavage.

The invention also relates to nucleic acids obtainable by the above methods. Preferably the invention relates to nucleic acids obtained by the above methods. Thus, in another aspect, the invention provides a nucleic acid obtained as described above.

In another aspect, the invention provides a nucleic acid vector comprising an origin of replication and a nucleic acid as described above. Preferably the origin of replication is capable of functioning in the target host eg. a prokaryote such as E. coli, a eukaryote eg. yeast such as P. pastoris, or other target organism. Preferably the origin or replication is a bacterial origin of replication capable of functioning in E. coli.

The origin of replication may be any suitable origin known to the person skilled in the art. Multiple origins of replication may be incorporated for different species, advantageously allowing shuttling between such species. Preferably the vector comprises a prokaryotic origin of replication such as those from plasmids R1 and R100 or others.

In another aspect, the invention provides use of a nucleic acid sequence comprising TAH for gene expression in the presence of Kid/PemK endoribonuclease, wherein said sequence does not contain TTACT.

In another aspect, the invention provides use of a nucleic acid sequence comprising TAC for gene expression in the presence of Kid/PemK endoribonuclease, wherein said sequence does not contain TTACT.

In another aspect, the invention provides use of a nucleic acid sequence comprising NTACN for gene expression in the presence of Kid/PemK endoribonuclease, wherein said sequence does not contain TTACT.

In another aspect, the invention provides use of a nucleic acid sequence comprising N1TACN2 for gene expression in the presence of Kid/PemK endoribonuclease, wherein at least one of N1 and N2 is V. V represents A or G or C, but not T. Thus, by ensuring that the core TAC sequence is not flanked on both sides by T, then the Kid/PemK recognition site is removed. Either side may independently be T, such as TTACV or VTACT, so long as both are not simultaneously T ie. so long as the sequence TTACT is not present, which sequence will be cleaved by Kid/PemK in RNA form.

According to the prior art, a sequence comprising TAH (UAH) will be digested by Kid/PemK. Therefore, based on the prior art, a sequence comprising this sequence would be unusable in the presence of Kid/PemK. However, the present invention provides that this sequence surprisingly can be used, provided it does not comprise the Kid/PemK recognition site TTACT. Preferably said sequence has been mutated to eliminate any occurrences of TTACT.

Preferably the present invention encompasses screening and/or mutation of sequences so that they do not contain TTACT (UUACU) and also do not contain CTACT (CUACU).

In another aspect, the invention provides a method for making a vector comprising selecting nucleic acid components for inclusion in said vector, screening the nucleotide sequence of said components for the sequence TTACT, wherein if one or more TTACT sequences is found then said sequence is mutated such that there are no longer any occurrences of TTACT, and assembling the nucleic acid components to produce the vector.

Of course, it will be appreciated by the skilled reader that assembly of individual sequences each devoid of TTACT could still lead to a vector comprising TTACT by joining of the junctional sequences. Thus, preferably the nucleic acid sequence of the final vector is itself screened and any TTACT sequences found are also removed by mutation.

Clearly, Kid/PemK endoribonuclease only acts on RNA. Thus, elements of the vector which are never intended to be transcribed into RNA need not be the subject of screening and/or mutation to remove potential TTACT sites. Thus, in a preferred embodiment, only regions or components of the vector which are intended to be, or be transcribed into, RNA are screened and/or mutated to eliminate TTACT sites. Preferably only regions or components of the vector which are intended to be, or be transcribed into, RNA such as dsRNA or ssRNA, preferably single stranded RNA (ssRNA), are screened and/or mutated to eliminate TTACT sites.

Preferably the vector comprises DNA.

In another aspect, the invention provides use of recombinant or purified Kid/PemK endoribonuclease for the cleavage of RNA comprising the sequence UUACU. Preferably the Kid/PemK endoribonuclease cleaves at the sequence UUACU. In one embodiment, the Kid/PemK endoribonuclease cleaves between the UU and the ACU parts of said UUACU sequence. In another embodiment, it may be that the Kid/PemK endoribonuclease cleaves between the UUA and the CU parts of said UUACU sequence. Preferably the Kid/PemK endoribonuclease cleaves between the UU and the ACU parts of said UUACU sequence.

In another aspect, the invention provides a method of inhibiting cell growth comprising inducing RNA cleavage by Kid/PemK at the sequence UUACU in said cell.

Preferably said RNA cleavage is induced by genetic or polypeptide delivery of Kid/PemK activity, preferably by genetic delivery.

Kid/PemK activity may be induced by delivery (such as genetic delivery) of Kid/PemK protein, or by downregulation of Kis/PemI antitoxin activity, for example where Kid/PemK is already present but its activity is suppressed by presence of the antitoxin. Preferably said genetic delivery is by gene therapy.

Downregulation of Kis/PemI may be at the protein and/or RNA level. Preferably the regulation is at the RNA level for example by inhibiting or down-regulating transcription, or by targeting the Kis/PemI transcript eg. for degredation.

When the cell is a eukaryotic cell such as a human cell, preferably inhibiting cell growth in this manner induces cell death. Preferably such cell death is via apoptosis.

In another aspect, the invention provides a method of inducing apoptosis in a eukaryotic cell said method comprising causing cleavage of RNA at UUACU site(s) in said cell. Preferably said cleavage is mediated by Kid/PemK.

In another aspect, the invention provides a method of making a ribonucleic acid resistant to Kid/PemK endoribonuclease, said method comprising

(a) providing a nucleic acid;
(b) screening the nucleic acid for the nucleotide sequence UUACU or TTACT;
(c) mutating said sequence such that there are no longer any occurrences of UUACU or TTACT;
wherein when the nucleic acid of (a) is a deoxyribonucleic acid, said method further comprises
(d) transcribing said deoxyribonucleic acid to produce ribonucleic acid.

In another aspect, the invention provides a method for downregulating expression of a nucleic acid in a system comprising introducing into said nucleic acid at least one TTACT or UUACU sequence, and inducing Kid/PemK activity in said system. Introducing may mean inserting or may mean mutating existing sequence in order to introduce at least one TTACT or UUACU sequence therein.

In another aspect, the invention provides a method for making a vector comprising selecting nucleic acid components for inclusion in said vector, screening the nucleotide sequence of said components for the sequence TTACT, wherein if no TTACT sequence is found then said nucleotide sequence is mutated such that there is at least one occurrence of TTACT, and assembling the nucleic acid components to produce the vector.

In another aspect, the invention provides a method of making a ribonucleic acid sensitive to Kid/PemK endoribonuclease, said method comprising

(a) providing a nucleic acid;
(b) screening the nucleic acid for the nucleotide sequence UUACU or TTACT;
(c) mutating said sequence such that there is at least one occurrence of UUACU or TTACT;
wherein when the nucleic acid of (a) is a deoxyribonucleic acid, said method further comprises
(d) transcribing said deoxyribonucleic acid to produce ribonucleic acid.

In another aspect, the invention provides a method for downregulating expression of a nucleic acid comprising at least one TTACT or UUACU sequence in a system, said method comprising inducing Kid activity in said system.

The system is preferably a cell or an in vitro system. Preferably the nucleic acid is a recombinant nucleic acid. Preferably the nucleic acid is a ribonucleic acid.

In another aspect, the invention provides a method for inducing or enhancing expression of a nucleic acid comprising at least one TTACT or UUACU sequence in a system comprising Kid activity, said method comprising reducing Kid/PemK activity in said system. Preferably said Kid/PemK activity is reduced by providing Kis/PemI.

In another aspect, the invention provides a method of induction of exoribonucleolytic degradation of a cistron 5′ of a UUACU site of an RNA molecule, comprising causing cleavage of said UUACU site. Preferably said cleavage is caused by inducing Kid endoribonuclease activity. Preferably said Kid endoribonuclease activity is induced by modulating Kid expression. Preferably said Kid endoribonuclease activity is induced by modulating Kis expression.

In another aspect, the invention provides use of recombinant or purified Kid/PemK to increase plasmid copy number in a system such as a cell.

In another aspect, the invention provides use of recombinant or purified Kis/PemI to increase plasmid copy number in a system such as a cell.

In another aspect, the invention provides use of Kid to reduce plasmid copy number in a system such as a cell, wherein UUACU sites have been introduced into one or more gene(s) involved in plasmid replication or maintenance. Preferably said gene is RepA. In this embodiment, UUACU sites are introduced into RepA so that induction of Kid expression results in degradation of RepA and thus reduces plasmid copy number, and/or prevents copy number increase.

In this embodiment, the invention may be advantageously applied to any plasmid which does not have a UUACU/TTACT site within an RNA which is required for plasmid replication or maintenance, such as antibiotic markers and stability systems. If it is desired to use this technique on a plasmid which does comprise such a site in an RNA needed for plasmid replication or maintenance, such as antibiotic markers and stability systems, then is it a simple matter to screen and mutate to destroy the site as described in detail herein.

In this embodiment, it is particularly advantageous when the plasmid encodes a Kid-resistant factor required for plasmid replication, like RepA in the case of plasmids R1 and R100, since RepA RNA contains no UUACU/TTACT sites and so will continue to be synthesised even in the presence of Kid/PemK.

Advantageously, partial Kid activity may be used. This permits cells to grow (albeit slowly), but any plasmid therein will increase its copy number (provided it is on the appropriate origin of replication; preferably it is on oriR1 or oriR100; preferably oriR1.). Thus, the invention can advantageously be applied to the manipulation and/or study of gene dosage by this technique of varying plasmid copy number using Kid/PemK. Furthermore, by using other (further) plasmids on origins which are Kid-insensitive, relative gene dosage effects can be dissected. In a preferred embodiment, this manipulation is performed using thermosensitive kis mutant(s). In particular, the thermosensitive kis mutant which is improperly folded at 37° C. and above and does not inhibit Kid at this temperature can be employed (preferably the kis mutant ‘kis17’ is used); by moving the cells to 30° C., some inhibition is produced, but this mutant is leaky, and at 30° C. Kid is not neutralized completely by the thermosensitive mutant Kis. Thus, at this temperature, cells grow more slowly than controls (with wildtype Kis and Kid) and the copy number of R1 increases. These applications of the invention are particularly valuable in the commercial production of nucleic acid molecules, since they enable a greater proportion of the total mass of the cell to be made of the target nucleic acid molecules. This finds application in techniques such as minicircle production. Briefly, this is the use of plasmids bearing (for example) two recombinase sites which are then induced to recombine, looping out unwanted sequence (for example the bacterial selectable marker and origin) and leaving the desired sequences on a separate recoverable circle of nucleic acid. By applying the present invention to amplify copy number before recombinase induction, the proportional mass of product obtained can be advantageously increased compared to techniques which do not involve a copy number amplification step. Furthermore, a greater copy number at the time of induction of recombinase activity leads to a more efficient production process, independent of the benefit of increased yields by mass. The invention also relates to a method for amplifying copy number comprising inducing Kid/PemK activity in a system such as a cell. This activity may be induced by inhibition or reduction of PemI/Kis activity. This aspect of the invention is itself surprising by comparison to the prior art since the prior art regarded Kid/Kis to be part of a post-segregational killing system. However, it is surprisingly disclosed herein that cells are not killed, but enter a recoverable stasis, and furthermore partial Kid activity, for example using temperature sensitive or ‘leaky’ Kis mutants, can advantageously be used to enhance copy number in living cells.

In summary, in contrast to what had been described previously, the inventors have demonstrated that the prokaryotic toxin Kid is an endoribonuclease that cleaves RNA specifically at 5′-UUACU-3′ sites. It is shown that Kid is part of a copy number rescue system in plasmid R1. When copy number of this plasmid decreases, Kid becomes active and selectively shuts off gene expression in the host cell, inhibiting its proliferation. Inhibition of protein synthesis by Kid is selective, as only the expression of genes containing TTACT sites is affected. As RepA, the limiting factor for R1 replication, does not contain any of these sites, it can be translated in the presence of Kid. This increases the replication rate of R1 and rescues the plasmid copy number.

Once the copy number of R1 is restored, a specific antitoxin, Kis, neutralizes Kid. Our work not only shows that inhibition of protein synthesis by Kid is selective, but also that Kid cleaves RNA at the pentanucleotide 5′-UUACU-3′, rather than the shorter, less specific trinucleotide 5′-UA(A/C/U)-3′ sequences described in the prior art. These are some of the advantages of the present invention.

### DETAILED DESCRIPTION OF THE INVENTION

### Kid/Kis

Kid cleaves specific mRNAs at UUACU sites. This surprising finding is exploited in the present invention. Without wishing to be bound by theory, in a biological context, it is thought that Kid acts to rescue the copy number of plasmid R1.

Stability and copy number of extra-chromosomal elements are tightly regulated in prokaryotes and eukaryotes. Toxin Kid and antitoxin Kis are the components of the parD stability system of prokaryotic plasmid R1 and they can also function in eukaryotes. In the prior art in bacteria, Kid was thought to become active only in cells that lose plasmid R1 and to cleave exclusively host mRNAs at UA(A/C/U) trinucleotide sites to eliminate plasmid-free cells. However, we demonstrate here that Kid becomes active in plasmid-containing cells when plasmid copy number decreases, cleaving not only host—but also a specific plasmid-encoded mRNA at the longer and more specific target sequence UUACU. This specific cleavage by Kid inhibits bacterial growth and, at the same time, helps to restore the plasmid copy number.

Kid cleaves a plasmid RNA that encodes a repressor of the synthesis of an R1 replication protein, resulting in increased plasmid DNA replication. This mechanism resembles that employed by some human herpesviruses to regulate viral amplification during infection. Thus, the present invention may advantageously be applied in similar context.

In the context of the present invention, ‘Kid resistance’ or ‘Kid-proof’ in connection with a nucleic acid means that that nucleic acid, or an RNA transcribed from it, will not be cleaved by Kid endoribonuclease. This resistance to cleavage may be assayed in absolute terms by nucleic acid analysis, or may be assayed functionally by permission of polypeptide expression from the nucleic acid of interest. In the context of the present invention preferably the test is permission of polypeptide expression. More preferably the test is absence of cleavage by assay of nucleic acids, most preferably the absence of Kid cleavage of the nucleic acid of interest, or RNA derived from the nucleic acid of interest. In a most preferred embodiment, Kid resistance or Kid proof describes a nucleic acid which has been mutated to remove all occurrences of TTACT (UUACU).

### Plasmid Maintenance

R1 is a low copy number plasmid of Escherichia coli which has been studied intensively. Its stable maintenance in bacterial cells is very sensitive to copy number fluctuations. Thus, R1 has evolved different genetic strategies to respond efficiently to these changes and to increase its stability in the host cell.

The initiation of R1 DNA replication is tightly controlled. The limiting factor for initiation of R1 replication at the origin is the initiator protein RepA. Its gene can be transcribed from two different promoters, PrcopB and PrrepA. When plasmid copy number is normal, a bicistronic transcript is synthesized. The upstream gene product of this mRNA, CopB, binds to the stronger promoter PrrepA and represses it, limiting plasmid DNA replication. An antisense RNA, copA, limits the translation of RepA, being less effective when PrrepA is fully active. If copy number of R1 decreases, the concentration of the repressor CopB also decreases, increasing the amount of repA-mRNA and therefore the copy number of R1 (see FIG. 1).

R1 also contains partition and post-segregational killing systems that act co-ordinately to reduce plasmid loss to frequencies below 10⁻⁷. One of these systems, parD, is located immediately downstream of the basic replicon of R1 (see FIG. 1). parD encodes a toxin (Kid; 12 kDa) that inhibits proliferation of plasmid-free daughter cells, and an antitoxin (Kis; 10 kDa) that protects plasmid-containing cells. In cells containing R1, Kis and Kid form a complex that neutralizes toxicity of Kid. Protease Lon degrades Kis, thereby triggering toxicity of Kid. However, the coordinate action of several regulatory loops allows the production of enough antitoxin Kis to keep toxin Kid neutralized when R1 is present. If the plasmid is lost during cell division, this balance shifts towards toxicity and Kid eliminates plasmid-free daughter cells.

It is surprisingly disclosed herein that the function of Kid is exerted in plasmid-containing cells, and not after plasmid loss. We demonstrate that Kid becomes active and inhibits bacterial growth if copy number of R1 decreases. Moreover, we show that Kid not only targets host mRNAs but also plasmid-encoded mRNAs, cleaving them specifically at the pentanucleotide 5′-UUACU-3′ rather than the less specific trinucleotide 5′-UA(A/C/U)-3′ as taught in the prior art. This 5′-UUACU-3′ is a more complex target sequence than previously described, allowing for greater specificity for individual RNA molecules.

In a biological context, when copy number of R1 decreases, Kid cleaves two of these UUACU sites in the intercistronic region of plasmid-encoded copB-repA-mRNA. This inhibits further synthesis of CopB and de-represses PrrepA. As a consequence, more monocistronic repA-mRNA is produced, resulting in more plasmid DNA replication, and the copy number of R1 is restored. We show that Kid also cleaves host-encoded mRNAs, such as dnaB and lon, specifically at 5′-UUACU-3′ sites. Thus, by acting simultaneously on host- and plasmid-encoded mRNAs, Kid inhibits cell growth and, at the same time, increases the copy number of R1.

Our results establish an important functional link between parD and the basic replicon of plasmid R1. They show that Kid contributes to plasmid stability by acting as a rescue system when R1 copy number decreases. Moreover, they suggest that plasmid R1 and several pathogenic eukaryotic viruses use similar molecular mechanisms to inhibit protein synthesis in their hosts and simultaneously increase their copy number. Thus, the invention advantageously enables systematic targeting of pathogenic eukaryotic viruses, for example to inhibit their protein production and restrain their replication.

Interestingly, Kis and Kid can also function in eukaryotes, and have been used to conditionally regulate cell proliferation and cell death in these organisms. This has biomedical and biotechnological relevance, as these proteins can now be used to develop strategies for the targeted elimination of tumor cells or specific cell lineages during development. Thus, a better understanding of how these proteins work also facilitates selective ablation of eukaryotic cells.

### Nucleotide Codes

Throughout this specification, the IUPAC codes are used. Specifically, the guidance of the Nomenclature Committee of the International Union of Biochemistry is followed. Thus, in this specification when discussing nucleotide sequences, A is used as an abbreviation for adenine; C for cytosine; G for guanine and T for thymine in DNA and uracil in RNA. It will be clear from the context in which terms are used whether nucleic acid generally (i.e. DNA and RNA) is being discussed, or whether a DNA or an RNA is being discussed. It should be noted that Kid endoribonuclease acts predominantly, and preferably only, on RNA. Thus, any references to cleavage in connection with TTACT (the DNA sequence) will preferably be understood to refer to cleavage of an RNA product generated from that DNA sequence. Furthermore, where a sequence features U for uracil then clearly this is referring to RNA.

Similarly, IUPAC codes for degenerate definition of nucleotide sequences are used. For example, His used to denote A or C or T but not G.

### Mutation

It is a key teaching of the present invention that TTACT sites are removed by mutation in order to produce RNA molecules which are resistant to the action of Kid endoribonuclease. Such mutation or engineering may be done by any suitable means known to those skilled in the art. For example, nucleic acids may be built from oligonucleotides, or may be synthesised directly by chemical means. Alternatively, mutation or engineering may be conducted by recombinant nucleic acid technology using PCR, site directed mutagenesis, ligation and cloning techniques and the like. As will be apparent to the skilled reader the method chosen to engineer the nucleotide sequences is a matter of choice for the operator working the invention. The most convenient method may vary according to circumstances, for example according to the number of mutations which need to be made or their spatial relationship to one another. The choice of technique for carrying out the engineering is well within the abilities of the skilled reader.

Preferably, mutations are silent with respect to the coding sequence. In other words, mutations made to the nucleic acid sequence will be chosen so that they do not alter the amino acid sequence encoded. If for some reason a nucleotide change has to be made which will affect the amino acid sequence encoded, then preferably a conservative amino acid substitution is made. Most preferably, mutation of the nucleotide sequence does not alter the encoded amino acid sequence (ie. ‘silent’ mutation).

As is well known to a person skilled in the art, a gene for directing protein expression may consist of many parts. For example, there may be an open reading frame (ORF), as well as an untranslated region (UTR) which itself may comprise a promoter and/or enhancer elements. Preferably, each element of a transcribed RNA is mutated so as to render it resistant to the action of Kid endoribonuclease. Preferably the RNA molecules of interest are altered by mutating the DNA template. In general, the fewer occurrences of TTACT there are, the better expression will be. Thus, it is preferred to reduce the number of occurrences of TTACT in the nucleotide sequence of interest. Preferably, these should be eliminated. Preferably, TTACT should be eliminated from the open reading frame being expressed. More preferably, TTACT should be eliminated from the open reading frame and promoter of the RNA being expressed. Most preferably, TTACT should be removed from the whole transcribed RNA involved in gene expression.

### Sensitivity to Kid Endoribonuclease

In some aspects of the invention, it may be desirable to create nucleic acid constructs which are sensitive to the action of Kid endoribonuclease. For example, it may be desired to shut off expression of a particular gene by inducing the expression of Kid endoribonuclease. In this embodiment, it is advantageous to include as many TTACT sites into the RNA involved in expression of the gene of interest as is possible. In this way, by inducing Kid activity, the maximum destructive effect will be wreaked upon the RNA whose expression is intended to be reduced or eliminated. Thus, the more Kid target sites there are within that RNA, the better the downregulation effect which will be achieved.

In working these embodiments of the invention, the considerations are the same as for the engineering of Kid resistance into genes of interest. Clearly, the difference is that Kid sites are incorporated rather than eliminated into the RNA of interest. Thus, techniques for mutating or engineering the RNA can be applied equally in these embodiments in order to produce RNA sequences having the desired number of TTACT target sites. Equally, the same considerations should be borne in mind when designing or engineering the constructs so that amino acid sequences are not altered by the incorporation of extra TTACT sites. These factors are well within the abilities of a person skilled in the art.

### Kid/PemK and Kis/PemI

As is known to a person skilled in the art, the Kid toxin from plasmid R1 is equivalent to the PemK toxin from plasmid R100. Similarly, the Kis antitoxin from plasmid R1 is equivalent to the PemI antitoxin from plasmid R100. Since these two toxin/antitoxin pairs are considered identical in the prior art, they are treated as interchangable for the purposes of the present invention. Thus, digestion by Kid endoribonuclease will be equivalent to digestion by PemK endoribonuclease. Similarly, neutralisation by Kis antitoxin will be equivalent to neutralisation by PemK antitoxin. Thus, embodiments of the present invention make use of Kid or PemK endoribonucleases interchangeably. The recognition site for these two equivalent enzymes is the same. Similarly, the invention relates to the Kis/PemI antitoxins as equivalents. Kid toxin may be neutralised by PemI or Kis. PemK toxin may be neutralised by PemI or Kis. Preferably, the toxin of the invention is Kid and the antitoxin of the invention is Kis.

The accession number for parD in Medline is X06240 or gi:45955. Gene kis corresponds to coordinates 784 to 1041 in the sequence shown. Kid gene corresponds to 1043-1375. It is to be noted that in the medline entry, Kid coordinates appear as 974-1375, due to the existence of two ATG start codons in frame with Kid inside the coding region for Kid. As the protein sequences were not known at the time that this entry was made, it was suggested therein that Kid was longer than it actually is. The correct coordinates are those given above. Thus, Kid protein starts with the downstream sequence ‘MERGE . . . ’ (rather than ‘MLKYQ . . . ’ as is suggested in the Medline entry). PemI and PemK have accession numbers P13975 and P13976 in the Swiss-Prot database.

### Single Protein Production (SPP)

Specific endoribonucleases can be used to achieve Single Protein Production (SPP) in living cells (Suzuki et al 2005 Mol. Cell, 18: 253-261). A SPP system has been developed recently using MazF, an endoribonuclease encoded by E. coli which cleaves mRNA at 5′-ACA-3′ sites. The activity of MazF arrests bacterial growth as it inhibits protein synthesis by depleting cellular mRNAs, as virtually all of them contain many 5′-ACA-3′ sites. However, any gene lacking 5′-ACA-3′ sites will be translated efficiently in vivo in the presence of MazF. Their protein products can be synthesized so efficiently under these circumstances (using the MazF-based SPP system), that they may constitute up to 90% of the total protein in the cell. A major disadvantage of the system is that almost every gene contains 5′-ACA-3′ sites. Moreover, most of them contain a large number of these sites, which need to be mutated (without altering the primary sequence of their protein product) before using them in the MazF-based SPP approach.

According to the present invention, Kid is a more advantageous endoribonuclease than MazF to for use in a SPP system. For example, comparison of the abundance of 5′-UUACU-3′ and 5′-ACA-3′ sites per gene demonstrates that using Kid instead of MazF advantageously requires very few mutations to be introduced in the gene to be solely expressed in vivo using a Kid-based SPP strategy (FIG. 8). This is illustrated in the following table which shows a comparison of the frequency with which 5′-TA(A/C/T)-3′,5′-ACA-3′ and 5′-TTACT-3′ are found in some prokaryotic and eukaryotic genes:

| | | | | | | | E6 |
|---|---|---|---|---|---|---|---|
| | repA | kid | trxA | GST | malE | EGFP A. | HPV |
| Sites | R1 | R1 | E. coli | E. coli | E. coli | Victoria | 18 |
| UA(A/C/U) | 19 | 9 | 9 | 38 | 52 | 14 | 28 |
| ACA | 11 | 3 | 3 | 8 | 25 | 16 | 16 |
| UUACU | 0 | 0 | 0 | 0 | 1 | 0 | 0 |

UA/A/C/U are the previously reported target sites for Kid (PemK).
ACA is the target site for MazF.
UUACU is the real target site for Kid (PemK), as disclosed herein.

A further comparison can be seen in FIG. 8.

For some genes, no mutation at all is required to render them Kid-resistant. This can be checked by screening for the UUACU (TTACT) site as explained herein.

SPP is preferably carried out according to the present invention as described in (Suzuki et al 2005 Mol. Cell, 18: 253-261), with the exception that Kid/Kis (or PemI/PemI) are used instead of MazF, and the nucleic acids of interest are mutated with regard to the true Kid/PemK cleavage site UUACU as taught herein.

### SOLO Strains

Instead of performing SPP according to Suzuki et al, other suitable strategies may be employed. For example, SOLO strains are preferred bacterial strains for use in SPP.

A SOLO strain is preferably any strain that carries in their chromosome (integrated) one or several elements required to perform SPP using Kid. These elements could be Kid itself, and/or Kis (eg. to regulate the system), and/or a repressor/inducer either resistant or sensitive to Kid activity that may be required. With these strains one should only need the regulated promoter and the gene to be expressed in a plasmid. Thus, SOLO strains advantageously offer additional flexibility in applications of the Kid based-SPP technology.

Thus, preferably SOLO strains contain elements of the SPP system integrated into the bacterial chromosome, advantageously allowing smaller plasmids to be used in the operation of SPP. Integration into the bacterial chromosome of gene(s) of interest is well within the ability of a person skilled in the art. Preferably a SOLO strain according to the present invention comprises one or more of the following integrated into the chromosome: inducible Kid, inducible Kis, gene of interest; preferably inducible Kid and inducible Kis, the inducible gene of interest being supplied extrachromosomally; preferably at least inducible Kid, (potentially making a single-use system in the absence of Kis for rescue), the inducible gene of interest being supplied extrachromosomally together with the inducible Kis (if required). The essential elements of SPP in the present invention are Kid and a Kid-resistant gene of interest for expression. Kis is a preferred optional component.

When using pSOLO™ plasmids, a preferred SOLO™ strain called GCM1 is advantageously used. GCM1 is strain DH4B of E. coli, with a copy of PrparD-kis integrated in its chromosome. This is particularly advantageous for use with the pSOLO plasmids that are regulated by anhydrotetracycline. The reasons for this are that Tet promoters are slightly leaky. Thus, basal expression of Kid takes place in the pSOLO plasmids even in the absence of Tet. Although minimal, this can be enough to inhibit the growth of, or even kill, E. coli cells. Having low expression of Kis from PrparD-kis integrated in the chromosome in GCM1 is enough to overcome this toxicity, whilst allowing SPP to work well upon induction of the Kid promoter in the pSOLO™ plasmids. Clearly, other pSOLO™ plasmids not depending on Tet may not benefit from this arrangement. Thus, preferably when the pSOLO™ plasmid uses a Tet promoter, preferably the host cell expresses low (i.e. compensatory) levels of Kis to counteract leakage of the Tet promoter; preferably the host cell is GCM1.

### Eukaryotic Single Protein Production (SPP)

Advantageously, the specificity of Kid and MazF is maintained in yeast and the effect of Kid in yeast cells is cytostatic and reversible which advantageously enables the use of a Kid-based SPP system in eukaryotic cells as well as in prokaryotic cells such as E. coli.

Extensive mutation of the genes of interest is required to eliminate all the 5′-ACA-3′ sites that they contain, before using them in the MazF-based prior art SPP system. However, since Kid cleaves RNA at 5′-UUACU-3′ sites and MazF at 5′-ACA-3′ sites, a SPP system based on Kid according to the present invention advantageously requires the introduction of very few mutations (if any) in the gene(s) of interest.

Moreover, Kid (and its antitoxin Kis) have been extensively used in eukaryotic cells in vivo and we disclose herein that both Kid and MazF maintain their cleavage specificities in these organisms. Thus, the present invention enables a Kid-based SPP system for eukaryotic cells. In such case, Kid would also be a better choice than MazF for the reasons explained above.

A Kid-based SPP system can be readily adapted to other existing technologies for protein expression/purification (e.g. kan^{r}, chlr^{r}, GST, Thioredoxin or EGFP do not contain UUACU sites, and MBP contains only one—see FIG. 8 and table above). Other genes, like tetR and amp^{r} may be appropriately mutated so that they lack 5′-TTACT-3′ sites (and so the RNAs will lack UUACU) as described herein.

Thus the essential elements of a SPP system according to the present invention are Kid polypeptide expression and a Kid-resistant gene of interest encoding the protein desired to be produced. Preferably the Kid-resistant gene is inducible. Preferably the Kid polypeptide expression is inducible. Alternatively the SPP system of the invention can be based on repressible Kis. For example, a strain can be constructed to constitutively express Kid and Kis, the Kis being repressible. In this scenario, the repression of Kis and induction of the gene of interest are preferably performed at approximately the same time, so that by repressing Kis function then Kid becomes active (no longer neutralised by Kis) and the Kid-resistant gene of interest is preferentially produced by SPP.

### Optimisation

The skilled worker may easily optimise the working of the invention for the particular application to which it is put eg. temperature sensitive protein production or similar application.

In particular, optimisation may focus on improving signal/background ratio. Examples of strategies for optimisation according to the present invention include
Different temperatures (e.g. 23° C.).
Different anhydrotetracycline (promoter inducer) concentrations
Different intercistronic Shine-Dalgarno sequences.
Introduction of intercistronic translation enhancer elements.
Split the bicistronic operon into two monocistronic operons.
Different strains can prove to be more efficient in the translation of eukaryotic codon usage.
Codon usage may be optimised to the strain (host cell) being used.

### VECTORS OF THE INVENTION

The invention also relates to vectors and/or strains allowing simultaneous expression of Kid and gene(s) of interest lacking the sequence 5′-UUACU-3′ in their mRNA(s). such vectors are referred to as pSOLO™ plasmids and SOLO™ strains. These plasmids/strains lack 5′-TTACT-3′ sites in genes relevant for plasmid replication, and/or required for proper maintenance (antibiotic markers), and/or replication (origin of replication/replication proteins), and/or regulation of transcription/translation (e.g. transcription factors) of the gene(s) of interest. Preferably each of these categories of gene lack TTACT in the plasmids/strains of the invention.

Advantageously, pSOLO plasmids according to the present invention can be configured to allow synthesis of tagged proteins (exemplary tag(s) may be one or more of FLAG, Strep tag, 6His, Maltose Binding Protein, Glutathione S-Transferase, Thioredoxin, liteins, EGFP, etc) as well as the use of different transcriptional regulators (TetR, IPTG, XylR, Nalidixic acid, Aspirin, Temperature, etc.) (eg. see FIG. 9 and example 10) in the presence of Kid/PemK activity by removing TTACT sites from the relevant ORFs, preferably from those listed above.

If required, DNA amplification of pSOLO plasmids can be achieved at the same time, using a oriR1 based vector, which replicates in the presence of Kid. The use of Kid/PemK in the enhancement of copy number is discussed in more detail below.

Similarly, the invention embraces pSOLO based vectors for use in eukaryotic cells such as yeast cells (eg. S. cerevisiae/P. pastoris). An additional level of regulation can be achieved through the action of Kis, the natural antitoxin for Kid, which can be included in the plasmid or the strain to be used as desired by the operator.

### Nucleotide Vectors

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably the vector is a plasmid vector. Preferably the plasmid vector is suitable for amplification in E. coli, preferably suitable for expression in E. coli. Preferably the plasmid vector is as shown in the accompanying drawings.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under conditions compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells may be used. This choice is well within the abilities of the skilled operator according to the context in which the vector such as pSOLO plasmid will be used. The promoter is typically derived from promoter sequences of viral or eukaryotic genes.

For example, it may be a promoter derived from the genome of a cell in which expression is to occur.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Vectors as described above contain promoters as outlined with respect to expression of the polynucleotide of interest. Furthermore, vectors of the invention preferably also comprise Kid and/or Kis sequences operably linked to promoters appropriate for their expression. In one embodiment, the nucleotide sequence for the polypeptide of interest and the nucleotide sequence for Kid (or Kis) are under the control of a single promoter i.e. these elements form a bicistronic operon (i.e. Kid upstream of gene of interest and regulated by a single common promoter). However, it is preferred that the nucleotide sequence for the polypeptide of interest and the nucleotide sequence for Kid (or Kis) are under the control of two separate promoters i.e. the arrangement is monocistronic (dual monocistronic). This arrangement has the advantage of allowing separate regulation of the polypeptide of interest and the Kid (or Kis) species. This may allow control of relative expression levels, relative expression kinetics or timing, or may allow independent switching of the production of the separate elements. This advantageously allows optimisation of the system, control of background expression and related features. In some embodiments it may even be desired to place the nucleotide sequence for the polypeptide of interest and the nucleotide sequence for Kid (or Kis) on separate vectors (e.g. separate plasmids).

Thus preferably a vector according to the present invention comprises a nucleotide sequence produced as described herein, an origin of replication, and one or more of Kis and Kid, preferably Kid. Preferably the nucleotide sequence produced as described herein and the Kid (or Kis) elements are under the control of separate promoters i.e. preferably the vector further comprises at least two promoters, at least one governing the expression of the nucleotide sequence produced as described herein and another governing the expression of the Kid (or Kis) element.

### Host Cells

Vectors and polynucleotides of the invention may be introduced into host cells for the purpose of replicating the vectors/polynucleotides and/or expressing the proteins of the invention encoded by the polynucleotides of the invention. Although the proteins of the invention may be produced using eukaryotic cells, for example yeast, insect or mammalian cells, in particular mammalian cells it is preferred to use prokaryotic cells as host cells, such as E. coli cells.

Vectors/polynucleotides of the invention may be introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### FURTHER APPLICATIONS

The invention can be applied in the production and purification of proteins in both basic and translational research.

Cells of the invention are preferably eukaryotic or prokaryotic cells, preferably eukaryotic cells.

The invention facilitates the construction of CHIPS (eg. onco-CHIPS).

The invention facilitates the construction of protein/peptide libraries, for example by excluding TTACT from said libraries and using them in the presence of Kid to obtain selective expression of said Kid-resistant libraries.

The invention finds application both in E. coli and in eukaryotic systems such as yeasts, for example S. cerevsiae and P. pastoris.

The invention may be applied in bacterial vector therapies.

The insights on which the present invention is based derive from an understanding of the behaviour of Kis/Kid inside cells rather than in cell free systems and thus preferably the systems of the present invention are used inside cells (preferably in vivo) rather than in cell free in vitro systems.

The invention may be used to enhance and/or produce metabolically viable bacteria/yeast ghosts eg. for bioreactor applications.

The invention may also be used in bacterial vaccines and protein/DNA bacterial vectors in therapy.

The invention may be applied in sensitisation to Kid, for example by mutating a nucleic acid resistant to Kid to introduce UUACU sites so that it becomes sensitive. Preferably such mutations are silent with respect to the genetic code. This embodiment is advantageous for the regulation of a gene of interest by using Kid, most preferably for down-regulating protein production from a gene of interest by rendering it Kid sensitive and introducing Kid into the system in order to degrade its RNA and thus down regulate its expression.

It should be noted that ‘single’ protein production in ‘SPP’ refers to the advantageous reduction of background protein expression and does not mean that only a single (i.e. one) protein of interest may be produced. Multiple proteins of interest may be produced in a single SPP if desired e.g. if making a multiprotein complex then each protein could be simultaneously produced in a single SPP system). These may even be produced from the same multicistronic transcript if desired.

It should be noted that the invention is not confined to the precise sequences of Kis/Kid given herein. As will be understood by the skilled reader, mutants or variants of Kid and/or of Kis that retain toxicity (Kid) and neutralization ability (Kis) are also embraced by the terms ‘Kid’ and ‘Kis’. The generation or isolation of such mutants is well within the ability of the person skilled in the art. In particular, the experimental detail provided herein allows the activities to be comprehensively assayed and tested so it is absolutely straightforward to determine whether or not a particular Kid or Kis mutant (or variant) retains its activity as required by the invention. For the avoidance of doubt, it is activity against UUACU (for Kid) or against Kid action (for Kis) which is important.

The present invention will now be described by way of example, in which reference will be made to the following figures:

### BRIEF DESCRIPTION OF THE FIGURES

FIG. 1 shows a diagram of oriR1 and parD loci. The initiator protein RepA can be transcribed as a monocistronic mRNA from promoter repA (PrrepA) or as a copB-repA bicistronic mRNA from the weaker promoter copB (PrcopB). Protein CopB represses PrrepA. In addition, an anti-sense RNA (copA) binds to its complementary mRNA sequence (copT, not shown for simplicity) in the copB-repA- and repA-transcripts, and limits the translation rate of RepA. As PrrepA is stronger than PrcopB and transcription of copA is constitutive, its inhibitory effect is weaker when monicistronic repA mRNA is being produced from PrrepA, which allows recovery of R1 copy number when CopB decreases. Transcription from promoter parD (PrparD) generates kis-kid bicistronic mRNAs. Antitoxin Kis binds to toxin Kid and neutralizes it, but rapid turnover of Kis by the host protease Lon activates toxicity of Kid. oriR1 indicates the plasmid R1 replication origin.

FIG. 2 shows a diagram, a graph and a photomicrograph. Kid and Kis can be expressed from thermo-sensitive promoters to inhibit bacterial cell growth and protein synthesis conditionally. (2A) Scheme of the plasmids used to induce kid and kis expression at 42° C. cI857 encodes a transcription factor that represses transcription from Pr_{L} at 30° C. but not at 42° C. (2B) Expression of Kid, but not of Kid plus Kis inhibits bacterial cell growth, compared to a control strain carrying empty plasmids. (2C) Expression of Kid, but not of Kid plus Kis, inhibits protein synthesis in vivo, as seen by ³⁵S-Met incorporation.

FIG. 3 shows photomicrographs and nucleotide sequences. Kid cleaves mRNA at UUACU sites: (3A) Primer extension analysis of dnaB transcripts. dnaB mRNA produced in vitro was incubated with buffer (Ctrl), Kid or Kid and Kis proteins (left side of panel). Alternatively, dnaB was expressed in vivo, alone (Ctrl) or together with kid (kid) for 30 or 60 minutes (right side of panel). Upper and bottom images show the upstream and downstream 5′-UUACU-3′ sites in dnaB mRNA, respectively. (3B) Primer extension analysis of lon transcripts. Strains carrying mini-R1 derivatives with wild type parD (kiskid), a thermo-sensitive parD (kis17kid) or the thermo-sensitive parD plus an extra copy of wild type kis (kis17kid+kis) were grown at 42° C. for 30 minutes. The most downstream 5′-UUACU-3′ site in lon mRNA cleaved by Kid is shown. Relevant sequences in (3A) and (3B) are shown on their axis (see text for details), with the 5′ ends of Kid cleavage products denoted in bold. (3C) Sequences of dnaB and lon mRNAs covered by our primer extension analysis. Cleaved 5′-UUACU-3′ sites are in bold and non-cleaved 5′-UU(A/C/U)-3′ sites are underlined.

FIG. 4 shows nucleotide sequence, photomicrographs, and a bar chart. Partial activation of Kid leads to cleavage of the copB-repA mRNA, de-represses PrrepA and increases the copy number of R1: (4A) Sequence of the copB-repA mRNA intercistronic region. Open boxes and underlined sequences denote transcription start sites and cleavage sites, respectively. SDrepA represents the Shine-Dalgano sequence for repA. 5′ or 3′ ends of open reading frames are shown in bold. (4B) Primer extension analysis of the copB-repA intercistronic region in strains transformed with mini-R1 derivatives carrying (i) a wild-type parD (kid), (ii) a leaky parD mutant (kis17kid), (iii) the same parD mutant plus wild-type kis (kis17kid+kis), iv) a mutant parD with inactive kid (kiskid18), or (v) a wild-type parD, but lacking copB and PrcopB (ΔcopB). The sequence analyzed is indicated on the axis, and the downstream 5′-UUACU-3′ site in this region underlined. The primer extension product at this site is denoted with a black arrow on the image, with its 5′ end indicated in bold on the axis. Transcription start sites in ΔcopB are indicated with a white arrow on the image and with a bent arrow and a +1 on the axis. The additional primer extension product detected in the kis17kid sample is indicated with a grey arrow. (4C) Real-Time PCR analysis of the repA/copB ratio in samples i) to iii) from (4B). Histograms represent the average values from three independent experiments. (4D) E. coli co-transformed with mini-R1 derivatives carrying wild type (wt) or the leaky parD mutant (17) and a compatible co-resident plasmid (pVTRA) with kis (Kis) or without it (Ctrl) were grown at 30° C. Plasmid DNAs were recovered, linearized and analysed by agarose gel electrophoresis. Ethidium bromide staining is shown. Numbers at the bottom indicate relative copy number of the mini-R1 derivative with respect to the co-resident plasmid as determined by southern blot using specific radiolabelled probes. (4E) is as for (4D), but using a mini-oriC replicon as the co-resident plasmid.

FIG. 5 shows photomicrographs and graphs. Kid arrests bacterial growth and impedes plasmid loss when R1 copy number decreases. (5A) The relative amount of mR1wt, mR1Kid18 and mR1M3 plasmids (mR1) in samples grown for 4 h in the presence (copA) or the absence (Ctrl) of copA over-expression from a co-resident plasmid (pPrTs−). (5B) Proliferation curves of strains used in (5A), grown in liquid media in the absence of selection for the mini-R1 derivatives. (5C) Relative colony forming units (cfu) of samples over-producing copA analyzed in (5B). (5D) Relative number of cfu analyzed in (5C) that still containing the mini-R1 derivative.

FIG. 6 shows photomicrographs and a graph. Kid inhibits the synthesis of CopB and restores R1 copy number through de-repression of PrrepA. (6A) Primer extension analysis of the copB-repA intercistronic region in samples analyzed at the 4 h time point in FIG. 5B. The sequence of this region in mR1wt is shown on the axis, with its downstream 5′-UUACU-3′ site underlined. The primer extension product detected at this site is denoted with a black arrow on the image, with its 5′ end indicated in bold on the axis. Transcription start sites seen in the ΔcopB sample in FIG. 4B are indicated with a white arrow on the image and with a bent arrow and a +1 on the axis. Two experiments with different loadings and exposure times (short- and long-exposure) are shown for samples expressing copA (6B) Kid inhibits the synthesis of CopB from a bicistronic copB-repA mRNA with a wild type intercistronic region (wt) but not with a mutant intercistronic region (M3). When Kis and Kid are expressed at the same time (kiskid), this effect is neutralized and the synthesis of CopB returns to control values (Ctrl). Detection of Pyruvate kinase (Pyk) using specific antibodies served as loading control. (6C) Relative copy number of mR1wt, mR1Kid18 and mR1M3 in bacteria when synthesis of extra copA is induced for 2 and 4 hours (40° C.; left side of the dashed line), and subsequently repressed for another 2 and 4 h (30° C.; right side of the dashed line). Each experiment was performed at least three times. The average result of three different experiments is shown in (6C).

FIG. 7 shows a diagram. Kid is part of a rescue system that regulates the copy number of R1. Transcription from PrcopB produces low amounts of a bicistronic copB-repA mRNA. The CopB represses transcription from the stronger PrrepA, keeping the copy number of R1 low. Low levels of kis-kid mRNA are synthesized from PrparD to produce Kid and excess Kis. These proteins form a complex that neutralizes Kid toxicity and represses transcription from PrparD. Rapid turnover of Kis by the host protease Lon de-represses PrparD and further excess of Kis is periodically produced. This maintains the rescue system in a constant “alert” state. If copy number of R1 decreases rapidly, the equilibrium is broken towards toxicity, and activates the rescue system. In this situation, remaining Kid cannot be readily neutralized, and it cleaves host- and plasmid-encoded mRNAs at UUACU sites. This arrests bacterial growth and prevents plasmid loss. At the same time, this activity inhibits synthesis of CopB, resulting in transcription from PrrepA. As a consequence, the replication rate of R1 increases. While copy number recovers, an excess Kis is rapidly produced from de-repressed PrparD, which progressively neutralizes Kid to restore the equilibrium.

FIG. 8 shows nucleotide sequences which have been annotated to provide a comparison of the frequency with which 5′-TA(A/C/T)-3′,5′-ACA-3′ and 5′-TTACT-3′ are found in some prokaryotic and eukaryotic genes. UA/A/C/U are the target sites for Kid (PemK) taught in the prior art such as Zhang et al., 2004. They are labelled in bold red case in the text. ACA is the target site for MazF. It is labelled in bold black (and underlined) case in the text. UUACU is the natural or biologically relevant target site for Kid (PemK), as determined by the present inventors. The single UUACU site found in the text (in MBP) is labelled in blue on a red background.

FIG. 9 shows a diagram of a vector according to the present invention, pSOLO HS3F.

FIG. 10 shows diagrams of vectors according to the present invention.

FIG. 11A shows diagrams of vectors according to the present invention in comparison with pTET vectors; FIG. 11B shows growth curves; FIG. 11C shows blots and a photomicrograph of protein expression.

FIG. 12A shows diagrams of vectors according to the present invention both with and without UUACU (TTACT) sites in the gene of interest; FIG. 12B shows the effects on protein expression.

FIG. 13 shows photographs of protein production according to the present invention (single protein production or ‘SPP’) compared with pTET based protein production.

FIG. 14 shows photographs illustrating optimisation and operation of the invention at low temperatures.

FIG. 15 shows a photograph illustrating optimisation and operation of the invention demonstrating advantageously low background.

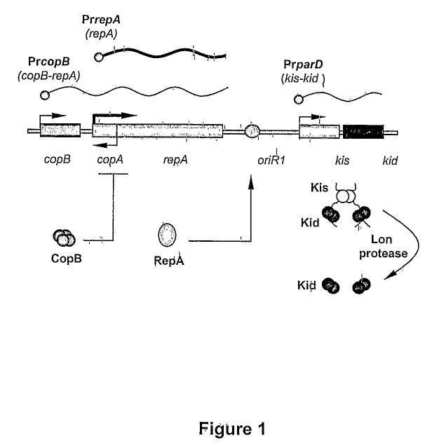

### EXAMPLES

The following materials and techniques are applied in the examples described below:

### Strains and Plasmids

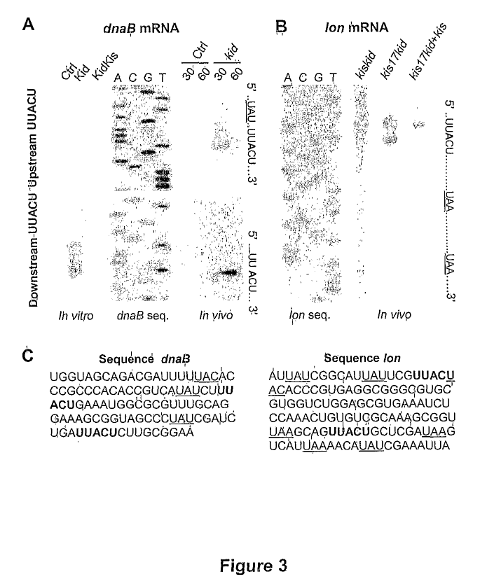

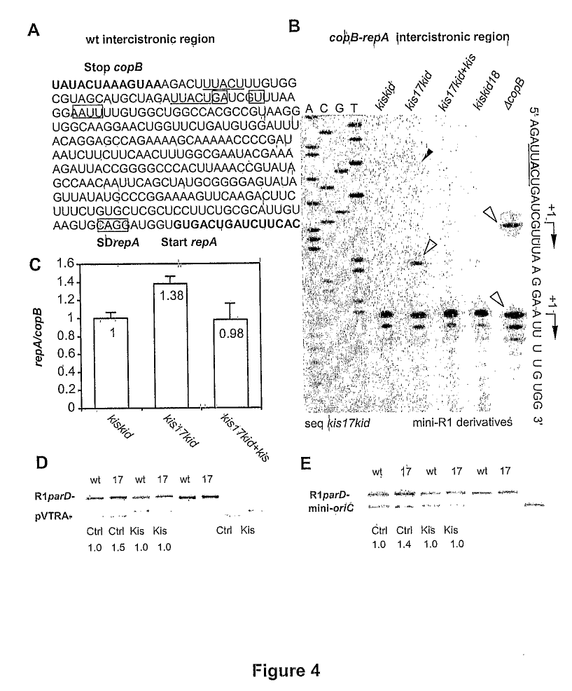

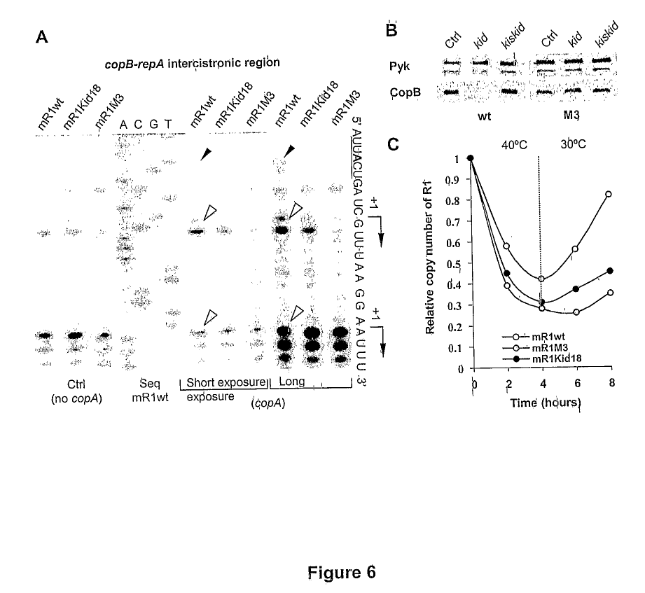

E. coli DH10B was used in all experiments. R1 plasmids with wild type-(pKN1562) and thermo-sensitive parD (pAB17) are as in Bravo et al. (1987 Mol Gel Genet vol 210 pp 101-110). The ΔcopB variant (pET80) is as in Ruiz-Echevarría et al (1995 FEMS Microbiol Lett vol 130 pp 129-136). Alternatively, the basic replicon of R1 and the parD system from these plasmids were amplified by PCR and ligated to the kan resistance gene (R1 derivatives in FIGS. 4B, 4C, 5, 6A and 6C). Kis was cloned in pPT150 Elvin et al (1990 Gene vol 87 pp 123-126) to produce pPrTsHCKis. The PstI-EcoRI fragment of pPT150 was cloned in pVTRA-A Pérez-Martín et al (1996 Gene vol 172 pp 81-86) to generate pPrTsLWC (PrTs, NC and LWC stand for thermo-sensitive promoter; high copy- and low copy plasmid, respectively). The same fragment was cloned between EcoRI and XmnI in pACYC184 to create p184PrTs. Kid was cloned in pPrTsLWC (pPrTsLWCKid). DnaB was cloned in pGADT7 (Clontech) and in pPrTsHC for the experiments in vitro and in vivo, respectively, in FIG. 3A. PrparD and kis were cloned in pVTRA-A to obtain pVTRAKis. The mini-oriC plasmid in FIG. 4E was obtained ligating a PCR fragment carrying oriC and its flanking mioC and gidA genes to the chlr resistance gene. A PCR product of copA was cloned in pPrTsHC for FIGS. 6A and 6C. Mutagenesis of the copB-repA intercistronic region (mR1M3) was made using oligos

(SEQ ID NO: 1)

5′CTAAAGTAAAGACTTTTCTTTGTGGCGTAGC3′

(SEQ ID NO: 2)

5′GCTACGCCACAAAGAAAAGTCTTTACTTTAG3′

(SEQ ID NO: 3)

5′GGCGTAGCATGCTAGATTTCTGATCGTTTTTGGAATTTTGTGGCTGGC
C3′
and

(SEQ ID NO: 4)

5′GGCCAGCCACAAAATTCCAAAAACGATCAGAAATCTAGCATGCTACGC
C3′.

To introduce the inactive kid18 mutant (Hargreaves et al 2002 Structure vol 10 pp 1425-1433) in mR1Kid18, oligos

### 5′CGCAGGTCATAAGCAGCAGGGAACGC3′ (SEQ ID NO: 5) and

### 5′GGCCGCGTTCCCTGCTGCTTATGACCTGC3′ (SEQ ID NO: 6)

were annealed and cloned into EcoNI and EagI of mR1wt. pKN1562 and mR1M3 were used as templates to amplify cmyc-copB-repA by PCR, which were cloned into p184PrTs for the experiments in FIG. 6B.

### Cell Growth and Protein Translation

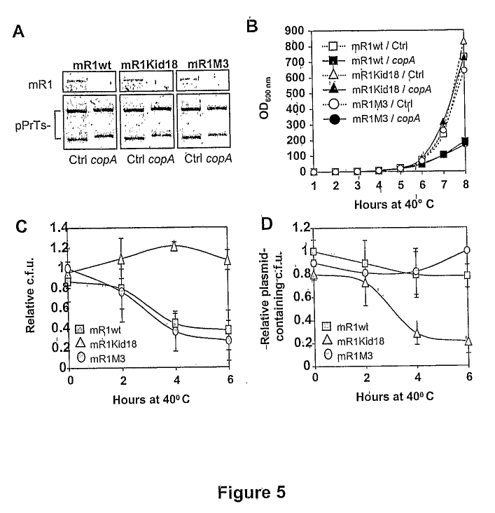

E. Coli transformed with pPrTsHC plus pPrTsLWC (Ctrl), pPrTsHC plus pPrTsLWCKid (Kid) or pPrTsHCKis plus pPrTsLWCKid (Kid/Kis) were grown in LB plus Amp (100 μg/ml) and Chlr (10 μg/ml) at 30° C. to an OD₆₀₀ of 0.2. Cultures were shifted to 42° C. and grown exponentially in pre-warmed medium. OD₆₀₀ was measured at the indicated time points. For the in vivo labelling experiments, 2 μCi of ³⁵S-Met were added to 500 μl of these cultures at the indicated times and incubated for 2 min before stopping the reactions with 10% TCA and 100 μg/ml of non-radioactive Met. Proteins were precipitated on ice for 1 hour, collected by centrifugation and analysed by SDS-PAGE. For the experiment in FIG. 6B, cells described above were co-transformed with p184PrTs-cmyc-copB-repA (carrying either wt or M3 intercistronic regions) and they were grown in LB plus Amp (100 μg/ml), Chlr (10 μg/ml) and Tet (10 μg/ml) at 30° C. until OD₆₀₀ was 0.4. Cultures were shifted to 40° C. for 1 hour and CopB expression was analyzed by Western Blot using monoclonal anti-c-myc tag antibody 9E10. Cells co-transformed with mR1wt, mR1Kid18 or mR1M3 and pPrTsHCcopA or pPrTsHC, and used in FIGS. 5, 6A and 6C, were grown exponentially at 40° C. in LB plus Amp (100 μg/ml). OD₆₀₀ was measured at time points indicated in FIG. 5B. At some of these time points, cells were plated in LB Amp and LB Amp/Kan and grown at 30° C. (FIGS. 5D and 5E). For FIG. 6A, temperature was shifted to at 30° C. after 4 hours of growth at 40° C. All experiments were performed at least three times.

### Primer Extension and Real-Time PCR

All primer extension and sequencing reactions were performed using the Primer Extension System AMV Reverse Transcriptase (Promega) and the Sequenase 2.0 (USB) kits, respectively, following manufacturer's instructions. A transcript spanning the first 465 nucleotides of dnaB was obtained using pGADT7DnaB digested with BglII as template and the MEGAscript kit (Ambion). 0.5 fmols of this RNA per sample were heated at 95° C. for 5 min in 20 mM Tris-Hcl, pH. 7.5; 75 mM NaCl and 10 mM MgCl₂ and allowed to reach room temperature. Samples were then incubated for 10 min at 37° C. with buffer or with Kid (4 pmols), either alone or with Kis (6 pmols). Proteins were purified as in de la De la Cueva-Méndez et al (2003 EMBO J. vol 22 pp 246-251). Samples were precipitated with ethanol and analyzed by primer extension using the oligo 5′-AGCAAAACCACCGACGCTATC-3′ (SEQ ID NO: 7).

DnaB was sequenced from pGADT7DnaB (FIG. 3A; left). Alternatively, the same analysis was performed using identical amounts of total RNA purified from cells carrying pPrTsHCDnaB and pPrTsLWCKid and cultured exponentially at 42° C. for the indicated times (FIG. 3A, right). For the analysis using lon, cells were grown exponentially at 42° C. for 30 min, and identical amounts of RNA extracted from them were used for each sample.

Oligo 5′-GGTTTTCGTTATCCGCGCGAC-3′ (SEQ ID NO: 8) was used for primer extension and sequencing reactions. A PCR product of lon was used as template for the sequencing reaction. For the analysis of copB-repA mRNA, cells were grown exponentially at 40° C. for the indicated times. Identical amounts of RNA from these samples were used. Oligo 5′-TAAATCCACATCAGAACCAGTT-3′ (SEQ ID NO: 9) were used for primer extension and sequencing reactions. In FIGS. 4B and 6A, loading of samples was adjusted so that the signal corresponding to the bottom white arrows had similar intensity. Real-Time PCR was performed in a DNA Engine OPTICON MJ Research, using SYBR Green Jump Start reagent (SIGMA).

### Oligo Pairs

| | |
|---|---|
| 5′-ATGTCGCAGAGAGAAAATGCAG-3′ | (SEQ ID NO: 10) |
| & | |
| 5′-CAGCGGCCATTTGTTTCTCAG-3′ | (SEQ ID NO: 11) |
| and | |
| 5′-GTGACTGATCTTCACCAAACGTAT-3′ | (SEQ ID NO: 12) |
| & | |
| 5′-GTTTTTCGCAGAACTTCAGCGT-3′ | (SEQ ID NO: 13) |

were used to amplify bicistronic- and monocistronic-repA cDNA, respectively. The repA/copB ratio was determined following the method described in Pfaffl (2001 Nucleic Acids Res vol 29 pp 2002-2007). All experiments were performed at least three times.

### Plasmid Copy Number

Samples in FIGS. 4D, 4E, 5A and 6C, were grown as indicated above. DNA purified from these samples was linearized, run in agarose gels, and stained with ethidium bromide (FIGS. 4D and 4E). For quantification, DNA from these gels was transferred to Zeta-Probe membranes (Bio-Rad) and probed with oriC-, pSC101ori-, and repA-(FIGS. 4D and 4E) or Amp^{r}- and parD-radiolabelled probes (FIGS. 5A and 6C). Labelling was performed with the Rediprime II labelling system (Amersham). Intensity of bands was quantified from X-ray films using a Fujifilm FLA-5000 densitometer. Relative copy number of the mini-R1 derivatives was determined using the intensity of the band corresponding to the co-existing plasmid as control reference. All experiments were performed at least three times.

### Example 1

### Kid Cleaves Host mRNA at UUACU Sites

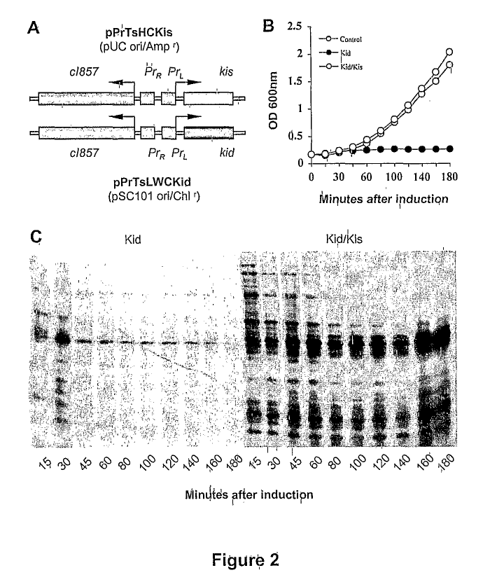

In this work we used thermo-sensitive promoters to regulate the expression of kis and kid independently (FIG. 2A). Induction of transcription from these promoters completely inhibited cell growth in bacteria containing only kid, but not in cells containing both kid and kis or control empty vectors (FIG. 2B). Protein synthesis was severely inhibited when transcription of kid was induced in exponentially growing cells and this effect was also neutralized when transcription of kis was induced at the same time (FIG. 2C).

PemK, the homologue of Kid in plasmid R100 is an endoribonuclease. We analysed whether Kid cleaves the host dnaB transcript, as this gene product had been previously implicated in the mode of action of Kid. Primer extension analysis showed that dnaB-mRNA is cleaved by Kid in vitro, and that this effect is inhibited when Kis is added to the reaction (FIG. 3A, left). Cleavage of dnaB-mRNA by Kid is also observed in vivo (FIG. 3A, right). Interestingly, in both cases Kid cleaved the dnaB-transcript at two different sites with identical sequence (5′-UUACU-3′; FIG. 3A, upper and bottom panels).

To examine whether Kid cleaves other host encoded transcripts with identical specificity we used a mini-R1 derivative carrying a thermo-sensitive mutation in the antitoxin gene (P18L; kis17) that inactivates it at 42° C. Total RNA was isolated from E. Coli transformed with the mini-R1 derivative and grown at 42° C. for 30 minutes before harvesting. Primer extension analysis of lon mRNA clearly showed that Kid cleaved this transcript in vivo (FIG. 3B). This activity was specific for Kid, as no cleavage was detected in cells co-transformed with a compatible plasmid expressing wild type Kis (kis17kid+kis) or with the control mini-R1 plasmid carrying wild type copies of kis and kid (kiskid) (FIG. 3B). Kid cleaved both dnaB and Ion mRNAs at identical sites (5′-UUACU-3′), highlighting the sensitivity of this sequence to the action of the toxin. Strikingly, although reported in the prior art as target sites for PemK, we did not detect cleavage in any of the adjacent 5′-UA(U/A/C)-3′ sites present in these mRNAs, apart from that embedded in 5′-UUACU-3′ (FIG. 3).

### Example 2

### Kid Cleaves Plasmid-Encoded copB-repA mRNA at UUACU Sites

We identified two 5′-UUACU-3′ sites in the copB-repA mRNA intercistronic region (FIG. 4A). This observation raised the interesting question of whether Kid also cleaves the bicistronic copB-repA mRNA. To analyze this, we took advantage of the leaky behaviour of the kis17kid mini-R1 derivative. In this plasmid mutant Kis has a reduced antitoxin activity at 30° C. Thus in bacteria containing it, cell growth is reduced 30% at 30° C. due to incomplete neutralization of Kid, although viability is not compromised (Bravo et al 1987 Mol Gen Genet vol 210 pp 101-110).

E. coli carrying this mini-R1 derivative was grown at 30° C. Primer extension analysis of the copB-repA intercistronic region showed that the downstream 5′-UUACU-3′ site was cleaved in this sample (FIG. 4B, black arrow). Longer exposure of the film showed that the upstream 5′-UUACU-3′ site in this region was also cleaved. These products were absent in control experiments where wild type Kis was co-expressed from another plasmid (FIG. 4B; kis17kid+kis) or using mini-R1 plasmids with wild type kis and kid (FIG. 4B; kiskid) or inactive kid (FIG. 4B; kiskid18).

### Example 3

### Kid Increases the repA/copB Ratio and the Copy Number of R1

Other primer extension products were identified in the samples analyzed in FIG. 4B. A mini-R1 derivative lacking copB and its promoter was used to reveal the transcription initiation sites of PrrepA (FIG. 4B; ΔcopB), which is de-repressed due to the absence of CopB. It showed that transcription from PrrepA initiates in a short region of 10 bp downstream of the 5′-UUACU-3′ sites (FIG. 4B; white arrows).

Another primer extension product was especially prominent in the kis17kid sample (FIG. 4B; grey arrow). Two observations suggested that this signal did not arise from cleavage of the copB-repA mRNA by Kid. First, although much weaker, this product was also detected in the control samples. Second, Kid did not cleave four identical sites (5′-UAA-3′) in the lon transcript (FIGS. 3B and 3C). Interestingly, that product lies in the short region from which PrrepA initiates transcription of monocistronic repA-mRNAs in the absence of CopB (FIG. 4B; ΔcopB), which suggested that PrrepA could be de-repressed in the kis17kid mini-R1 derivative.

The repA/copB ratio should increase when PrrepA is de-repressed. Real Time-PCR showed that the repA/copB ratio increases 38% in the kis17kid sample compared to the kiskid sample. Moreover, this ratio returned to control values when wild type kis was co-expressed in the kis17kid sample (kis17kid+kis) (FIG. 4C). These results suggest that the signal detected in our primer extension (FIG. 4B, grey arrow) corresponds to monocistronic repA-mRNA.

RepA is the limiting factor for initiation of replication in plasmid R1. Thus, when transcription from PrrepA is de-repressed, copy number of plasmid R1 increases. As the kis17kid sample has a higher repA/copB ratio, we measured the relative copy number of this mini-R1 derivative. At 30° C. the copy number of this plasmid is 1.5 fold higher than that of a kiskid control, relative to a co-resident plasmid (FIG. 4D; Control). This difference was abolished if wild type Kis was expressed from the co-resident plasmid (FIG. 4D; Kis), or if kid was deleted from kis17kid. Moreover, as antitoxin activity of Kis in the kis17kid mini-R1 decreases with temperature, the difference in copy number is almost 2 fold when cells were grown at 37° C. for 30 minutes before quantification. Similar results were obtained when a mini-oriC replicon was used as the co-existing compatible plasmid (FIG. 4E). Taken together, these results suggest that partial activation of Kid in a kis17kid R1 derivative increases the synthesis of repA and consequently the copy number of this plasmid.

### Example 4

### Kid Cleaves the copB-repA mRNA when R1 Copy Number Decreases

FIG. 4 shows that partial activation of Kid cleaves the copB-repA transcript and suggests that this cleavage is linked to de-repression of PrrepA. It also shows that partial activation of Kid increases the copy number of R1. Previous observations suggest that parD contributes effectively to plasmid stability only when replication of R1 is compromised. Thus, we decided to test whether increasing the intracellular concentration of copA, which inhibits the replication rate of R1, activates toxicity of Kid and leads to cleavage of the intercistronic region in the copB-repA mRNA.

We co-transformed E. coli with a plasmid expressing copA from a thermo-sensitive promoter or with an empty control plasmid, and a mini-R1 derivative carrying a wild type parD (mR1wt). As controls, we used mR1M3 (mR1wt with the 5′-UUACU-3′ sites in the copB-repA intercistronic region mutated to 5′-UUUCU-3′) and mR1Kid18 (mR1wt carrying a nontoxic Kid mutant). The relative copy number of these mini-R1 derivatives decreases at 40° C. when co-transformed with the copA expressing plasmid, compared to those co-transformed with the control empty plasmid (FIG. 5A). We also analyzed cell proliferation in these strains at 40° C. In the absence of antibiotic selection for the mini-R1 derivatives, bacterial growth was inhibited in cells carrying mR1wt or mR1M3, but not mR1Kid18, when transcription of copA was induced (FIG. 5B). These results demonstrated that inhibition of cell growth is due to activation of Kid, and that it only occurs when plasmid copy number decreases.

Next, we examined the contribution of Kid activation to plasmid stability in our experimental set up. The same experiment as in FIG. 5B was performed, but plating cells at 30° C. in either Amp (to determine the number of viable cells) or Amp/Kan (to measure the fraction of cells still containing plasmid R1) at different time points. The relative number of colony forming units (cfu) decreased with time in cells transformed with mR1wt and mR1M3, compared to those transformed with mR1Kid18 (FIG. 5C). However, the relative number of plasmid-containing cfu did not decrease with time for samples mR1wt and mR1M3, compared to sample mR1Kid18 (FIG. 5D). These results show that activation of Kid inhibits cell growth and impedes plasmid loss. Thus, activation of Kid occurs in plasmid containing cells, which suggest that Kid could cleave the copB-repA mRNA when copy number of R1 decreases.

To examine this, RNA was purified from samples grown as in FIG. 5, and the copB-repA intercistronic region was analyzed as before. Cleavage of both 5′-UUACU-3′ sites in this region was detected in the mR1wt sample when expression of copA was induced from the co-resident plasmid for 4 h (FIG. 6A, only the downstream 5′-UUACU-3′ site is shown, indicated with a black arrow). No cleavage was detected in any mR1Kid18 or mR1M3 samples, or in cells co-transformed with mR1wt and the empty control plasmid lacking copA. These results confirm that Kid becomes active in plasmid-containing cells if the copy number of R1 decreases. Moreover, they demonstrate that this leads to cleavage of the copB-repA intercistronic region specifically at its 5′-UUACU-3′ sites.

### Example 5

### Kid Inhibits the Synthesis of copB to Restore R1 Copy Number

FIG. 6A also shows that PrrepA is strongly de-repressed when copy number of mR1wt (but not of mR1M3) decreases. Interestingly, PrrepA of mR1Kid18 is slightly de-repressed in the same experiment (FIG. 6A). However, decreasing the copy number of mR1Kid18 neither inhibits bacterial growth (FIG. 5B), nor avoids plasmid loss (FIG. 5C), and is not linked to mRNA cleavage (FIG. 6A). These observations highlight the essential role that Kid and the UUACU sites in the copB-repA mRNA play for the correct functioning of the CopB rescue system.

As de-repression of PrrepA occurs when the concentration of CopB decreases, we examined the effects of Kid on the synthesis of CopB from the bicistronic copB-repA mRNA. We cloned the copB-repA ORFs from mR1wt and mR1M3 in a thermo-sensitive expression vector, and used them in the strains shown in FIG. 2. This allowed us to induce transcription of copB-repA mRNA and of kid or kid and kis mRNAs simultaneously. Western-blot showed that Kid significantly reduced the synthesis of CopB, and that Kis neutralizes this inhibition (FIG. 6B; wt). Moreover, the effect of Kid on CopB synthesis depends entirely on the integrity of the 5′-UUACU-3′ sites in the copB-repA-mRNA, as no inhibition was detected using the intercistronic mutant control (FIG. 6B; M3).

Our results demonstrate that Kid inhibits the synthesis of CopB from copB-repA mRNA. This explains why cleavage of copB-repA mRNA by Kid de-represses PrrepA (FIGS. 4B and 6A) and the higher copy number of kis17kid mini-R1 (FIGS. 4D and 4E). We compared the relative copy numbers of mR1wt, mR1Kid18 and mR1M3 in the experiment shown in FIG. 6A. Cultures were grown at 40° C. and, after 4 h, temperature was shifted to 30° C. to inhibit further transcription of copA from the co-resident plasmid. The relative copy number of each mini-R1 derivative was determined every 2 h by southern blot (FIG. 6C). Our results demonstrate that, when grown at 40° C., the relative copy number of mR1wt decreases more slowly than that of mR1Kid18 and mR1M3. Moreover, when further transcription of copA was repressed, the copy number of mR1wt was restored faster than that of mR1Kid18 and mR1M3 (FIG. 6C).

### Example 6

### Kid Cleaves Host- and Plasmid-mRNAs at 5′-UUACU-3′

The prior art discloses that over-expression of PemK, the homologue of Kid in plasmid R100, leads to cleavage of host mRNAs at 5′-UA(A/C/U)-3′ sites (Zhang et al., 2004). Host protease Lon is responsible for the rapid turnover of antitoxin Kis, and another host gene, dnaB, had been implicated in the mode of action of toxin Kid, although its involvement remained to be fully understood.

We analyzed the effects of Kid on dnaB and lon mRNAs and surprisingly showed that Kid cleaves both transcripts specifically at 5′-UUACU-3′ sites. Interestingly, no cleavage was observed at any of the ten adjacent 5′-UA(A/C/U)-3′ sites in these mRNAs (FIG. 3). Thus, our work reveals that Kid (thus, PemK) cleaves mRNA at 5′-UUACU-3′ sites, which represents a longer, more specific sequence than described in the prior art.

parD was identified as a post-segregational killing system contributing to the stable maintenance of R1 plasmid in bacteria. Post-segregational killing systems eliminate plasmid-free cells. However, we demonstrate here that Kid activation surprisingly occurs in plasmid-containing cells and also cleaves the copB-repA mRNA, specifically at 5′-UUACU-3′ sites (FIGS. 4B and 6A). This novel pre-segregational role of Kid is independent of ribosomes, as cleavage of the same 5′-UUACU-3′ sites is detected both in vivo and in vitro, using a reconstituted system (FIG. 3A). Furthermore, Kid cleaves the intercistronic (i.e. non-translated) copB-repA mRNA region in vivo, which suggests that its activity is not coupled to that of translation by ribosomes (FIGS. 4B and 6A).

Furthermore, it is disclosed herein that “de novo” synthesis of at least two proteins can take place in the presence of active Kid.

One is RepA of R1, which promotes restoration of the plasmid copy number. Analysis of repA mRNA reveals that it contains three 5′-TTACC-3′ sites (see sites underlined in ‘RepA of R1’ below). Thus, it is shown that TTACC (ie. equivalent to a silent mutation (frame 3) of TTACT to TTACC) is Kid resistant.

RepA of R1
GTGACTGATCTTCACCAAACGTATTACCGCCAGGTAAAGAACCCGAATCC
GGTGTTCACTCCCCGTGAAGGTGCCGGAACGCTGAAGTTCTGCGAAAAAC
TGATGGAAAAGGCGGTGGGCTTCACCTCCCGTTTTGATTTCGCCATTCAT
GTGGCGCATGCCCGTTCCCGTGGTCTGCGTCGGCGCATGCCACCGGTGCT
GCGTCGACGGGCTATTGATGCGCTGCTGCAGGGGCTGTGTTTCCACTATG
ACCCGCTGGCCAACCGCGTCCAGTGTTCCATCACCACACTGGCCATTGAG
TGCGGACTGGCGACAGAGTCCGGTGCAGGAAAACTCTCCATCACCCGTGC
CACCCGGGCCCTGACGTTCCTGTCAGAGCTGGGACTGATTACCTACCAGA
CGGAATATGACCCGCTTATCGGGTGCTACATTCCGACCGACATCACGTTC
ACACTGGCTCTGTTTGCTGCCCTTGATGTGTCTGAGGATGCAGTGGCAGC
TGCGCGCCGCAGTCGTGTTGAATGGGAAAACAAACAGCGCAAAAAGCAGG
GGCTGGATACCCTGGGTATGGATGAGCTGATAGCGAAAGCCTGGCGTTTT
GTGCGTGAGCGTTTCCGCAGTTACCAGACAGAGCTTCAGTCCCGTGGAAT
AAAACGTGCCCGTGCGCGTCGTGATGCGAACAGAGAACGTCAGGATATCG
TCACCCTAGTGAAACGGCAGCTGACGCGTGAAATCTCGGAAGGACGCTTC
ACTGCTAATGGTGAGGCGGTAAAACGCGAAGTGGAGCGTCGTGTGAAGGA
GCGCATGATTCTGTCACGTAACCGCAATTACAGCCGGCTGGCCACAGCTT
CTCCCTGA

The other gene is Kis. Another implication of our work is that the effect of Kid is reversible. For this to happen, Kis also needs to be synthesized “de novo” in the presence of active Kid. Thus, Kis must be resistant to the action of Kid. We examined the sequence of kis and show that it contains a 5′-CTACT-3′ (ie. equivalent to a silent mutation (frames 1 and 2) of TTACT to CTACT) which is underlined in ‘Kis of R1/PemI of R100’ below. Thus it is shown that CTACT is Kid resistant.

Kis of R1/PemI of R100
ATGCATACCACCCGACTGAAGAGGGTTGGCGGCTCAGTTATGCTGACCGT
CCCACCGGCACTGCTGAATGCGCTGTCTCTGGGCACAGATAATGAAGTTG
GCATGGTCATTGATAATGGCCGGCTGATTGTTGAGCCGTACAGACGCCCG
CAATATTCACTGGCTGAGCTACTGGCACAGTGTGATCCGAATGCTGAAAT
ATCAGCTGAAGAACGAGAATGGCTGGATGCACCGGCGACTGGTCAGGAGG
AAATCTGA

### Example 7

### Kid Activity in Plasmid-Containing Cells Increases Plasmid Copy Number

We used two different approaches to analyze the consequences of Kid activation in plasmid-containing cells. First, we used a leaky mutant parD in a wild type R1 replicon context. We show that incomplete neutralization of Kid in this mutant leads to cleavage of the intercistronic region in copB-repA mRNA, and to de-repression of PrrepA (FIG. 4B; kis17kid). Furthermore, we show that the repA/copB ratio increases 38% in this sample (FIG. 4C). Consequently, the relative copy number of this mini-R1 derivative increases 1.5 fold compared to controls (FIGS. 4D and 4E).

Using a mini-R1 plasmid with a wild type parD system (mR1wt), we demonstrate that Kid cleaves the copB-repA intercistronic region when plasmid copy number decreases (FIGS. 5 and 6A). This depends entirely on the presence of wild type kid and on the integrity of the sites targeted by Kid in the copB-repA mRNA, and it also leads to de-repression of PrrepA (FIG. 6A). Consequently, when copA is over-expressed from a co-resident plasmid, the copy number of in mR1wt remains higher than that of mR1Kid18 (with inactive Kid) or mR1M3 (lacking UUACU sites in copB-repA mRNA). Moreover, in the absence of further synthesis of copA, mR1wt restores its copy number faster than mR1Kid18 and mR1M3 (FIG. 6C).

We also addressed how the activity of Kid is linked to de-repression of PrrepA. Kid inhibits the synthesis of CopB from the bicistronic copB-repA mRNA, but not if the two 5′-UUACU-3′ sites in its intercistronic region are mutated (FIG. 6B). Therefore, inhibition of CopB synthesis when Kid cleaves the copB-repA mRNA explains how PrrepA is de-repressed. Cleavage of the copB-repA mRNA by Kid occurs downstream of the copB gene, as no other 5′-UUACU-3′ sites are found in this molecule. Endoribonucleolytic cleavage of polycistronic mRNAs often triggers exoribonucleolytic degradation of its upstream cistrons. Thus, it is shown that once Kid cleaves the intercistronic region of the copB-repA mRNA, upstream copB is degraded by exoribonucleases.

De-repression of PrrepA linked to copB-repA mRNA cleavage is observed using a leaky parD mutant and a wild type parD (FIGS. 4B and 6A). Transcription from PrrepA initiates within a 10 bp region located downstream of the 5′-UUACU-3′ sites in the copB-repA intercistronic region, as seen using a ΔcopB mini-R1 derivative (FIG. 4B; white arrows). De-repression of PrrepA observed using wild type parD occurs at the same transcriptional initiation site that is de-repressed in the absence of copB (FIG. 6A, upper white arrow). However, transcription of repA initiates at a slightly more downstream site when we use the leaky mutant parD (FIG. 4B, grey arrow). This is probably due to differences in the way parD behaves in each case. In wild type parD, Kid becomes active when R1 copy number decreases. In the leaky parD mutant, toxicity of Kid is partial and increases R1 copy number. Without wishing to be bound by theory, perhaps in cells carrying this mutant the intracellular level of CopB decreases, but remains higher than when Kid becomes active from a wild-type parD. This may determine the location of the initiation sites of repA-mRNAs within this region.

In kis17kid R1, transcription from de-repressed PrrepA starts in the sequence 5′-UAA-3′ (FIG. 4B, grey arrow). This sequence had been described in the prior art as a cleavage site for PemK. However, three experimental observations provide strong evidence that this signal corresponds to initiation of monocistronic repA-mRNA synthesis, and not to mRNA cleavage by Kid. First, we show that three identical sites (5′-UAA-3′) are not cleaved in lon mRNA by Kid (FIG. 3B). Second, that signal is also detected in the absence of Kid activation (FIGS. 4B and 6A). Third, we do not observe cleavage of this site activating Kid from wild type parD (FIG. 6A).

### Example 8

### Kid can be Used to Sense and Regulate Plasmid Copy Number

The prior art proposes that CopB has been kept by R1 to act as a rescue system in cells with very few copies of the plasmid. This proposal is based on the indirect observation that extra CopB increases the loss rate of R1 derivatives and strongly represses transcription of a reporter gene from PrrepA. We surprisingly demonstrate that, rather than acting post-segregationally as a killer system, Kid is in fact part of that copy number rescue system and has evolved to act pre-segregationally. Indeed, our work helps to explain unanswered questions relating to the sensitivity of this system if it depends exclusively on CopB. In cells where copy number of R1 decreases, activation of Kid leads to cleavage of host mRNAs at 5′-UUACU-3′ sites and this inhibits cell proliferation, which prevents plasmid loss during division (FIG. 5). At the same time, Kid cleaves the plasmid encoded copB-repA mRNA with the same specificity (FIG. 6A). This decreases the intracellular concentration of CopB (FIG. 6B) and de-represses PrrepA (FIG. 6A) stimulating recovery of R1 copy number (FIG. 6C). Although dilution of CopB may contribute to activating the rescue system when R1 copy number is very low, it has been acknowledged in the art that its sensitivity would be greater if CopB were actively degraded. We show herein that reducing the copy number of mR1wt and mR1M3 activates Kid and that this arrests bacterial growth and avoids plasmid loss (FIG. 5). However, PrrepA is not de-repressed in mR1M3 (FIG. 6A), which indicates that dilution of CopB in this situation is not enough to activate the rescue system from the plasmid. Partial de-repression of PrrepA is observed when the copy number of mR1Kid18 decreases (FIG. 6A). However, Kid is not functional in this plasmid and it neither inhibits bacterial growth nor avoids plasmid loss (FIG. 5). As a consequence, dilution of CopB can occur to an extent that allows partial de-repression of PrrepA, but this happens at the expense of great plasmid instability (FIG. 5D). This is further supported by comparison of FIGS. 5D and 6C. Relative number of c.f.u. containing mR1Kid18 (but not mR1M3) decreases after 4 h in FIG. 5D. However, relative copy numbers of mR1Kid18 and mR1M3 are similar at this time point (FIG. 6C). Thus, fewer cells still contain mR1Kid18 but, due to de-repression of PrrepA (FIG. 6A), they do so at higher copy numbers than those containing mR1M3 although this cannot prevent plasmid loss (FIG. 5D). Thus, our results reveal that it is only when functional Kid and UUACU sites in the copB-repA mRNA are present that the rescue system can operate efficiently. In this case Kid inhibits bacterial growth and new synthesis of CopB simultaneously, providing the right conditions to dilute CopB progressively without any plasmid loss. This eventually de-represses PrrepA and restores the plasmid copy number.

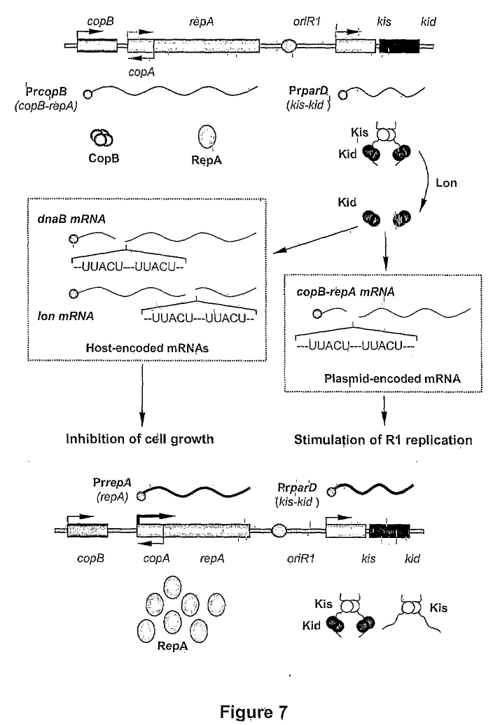

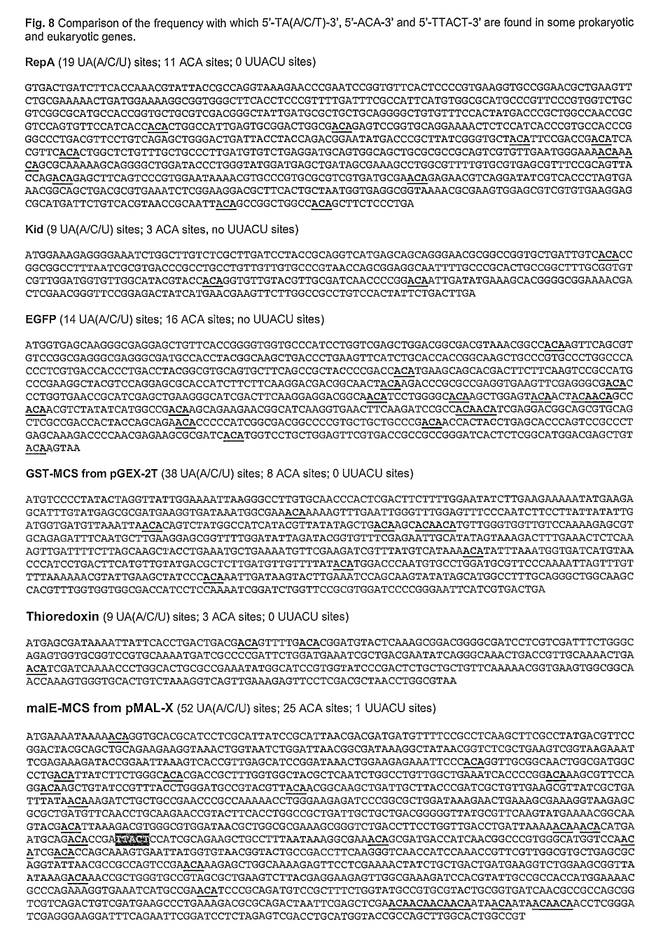

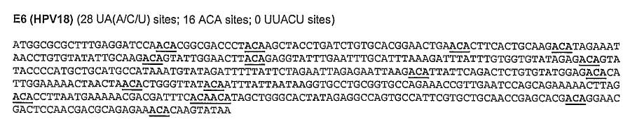

Based on these observations, we propose here a model to explain how parD acts pre-segregationally to stabilize plasmid such as R1 (FIG. 7). When copy number of R1 is normal, transcription from PrcopB produces both CopB and RepA. CopB represses transcription from PrrepA and limits the replication rate of R1. In this situation, transcription from PrparD is low. It produces Kis and Kid, which form a complex that neutralizes the toxin and represses PrparD. Thus, equilibrium is reached to maintain the copy number of R1 and the synthesis of Kid and Kis fairly low and constant. However, Kis is continuously degraded by the host protease Lon. Faster turnover of Kis is counteracted by several regulatory loops to ensure neutralization of Kid, but this balance is broken towards toxicity if copy number of R1 decreases rapidly. When this happens, degradation of Kis produces a relatively large amount of free Kid that cannot be readily neutralized by newly synthesized Kis from very few copies of the plasmid. This free Kid acts pre-segregationally, inhibiting or arresting cell growth and, simultaneously, decreasing the intracellular concentration of CopB and de-repressing PrrepA. As a consequence, replication rate of R1 increases and its copy number is rapidly restored. In the absence of enough Kis, PrparD is also de-repressed, and it produces more Kis than Kid. Thus, as copy number of R1 recovers, the intracellular concentration of Kis increases faster than that of Kid. This progressively neutralizes the effects of the toxin until the equilibrium is restored again (FIG. 7). Interestingly, the copB genes of plasmids R1 and R100 differ in sequence but not in function. Most interestingly, R100 conserves one of the 5′-UUACU-3′ sites cleaved by Kid in the copB-repA intercistronic region of R1. This observation strongly supports the view that the novel function of Kid disclosed herein is also relevant for the stable maintenance of R100 by PemK. Thus, it is shown that this system can be exploited according to the present invention to influence plasmid maintenance and/or amplification of plasmid copy number.

We investigated how activation of PrrepA translates into higher production of RepA, essential to increase the copy number of R1, given that Kid inhibits protein synthesis. Conspicuously, monocistronic repA-mRNAs do not contain 5′-UUACU-3′ sites (although they contain 19 sites with the sequence 5′-UA(A/C/U)-3′, described in the prior art as target sites of PemK). By cleaving mRNAs specifically at 5′-UUACU-3′ sites Kid helps to dilute CopB, de-repressing PrrepA without inhibiting de novo synthesis of RepA. This insight is exploited in applications of the present invention.

The teachings presented herein differ from prior art stating that PemK (or Kid) cleaves mRNAs with less specificity. Our work demonstrates that Kid has evolved to act exquisitely in plasmid-containing cells, cleaving host- and plasmid-encoded mRNAs at 5′-UUACU-3′ sites, and acting in a reversible manner to regulate the copy number of plasmid such as R1.

### Example 9

### The Function and Activity of Kid Resemble Viral Host Shutoff of Herpesviruses

Human herpesviruses have acquired the ability to alter both host and viral mRNA stability. The virion host shutoff protein (Vhs) drives this process through its endoribonucleolytic activity. During lytic infection Vhs accelerates the degradation of cellular mRNAs, leading to an overall decrease in host protein synthesis. Following the onset of viral transcription, Vhs accelerates the turnover of viral mRNAs. By shortening the half-lives of all mRNAs, Vhs redirects the cell from host to viral gene expression, facilitates the sequential expression of different classes of viral genes, and stimulates the replication of the viral genome.

Strikingly, the mode of action of these viral host shutoff proteins shares several features with Kid. They not only cleave host mRNAs, but also viral mRNAs and, in the latter case, they seem to target intercistronic regions. Furthermore, Vhs preferentially affects the stability of mRNAs with AU-rich elements (AREs), which contain repetitions of the sequence 5′AUUUA 3′. Thus, it seems that prokaryotic plasmids such as R1 and human herpesviruses may have evolved similar strategies to stimulate their own replication whilst shutting off host protein synthesis. Thus, the present invention can be advantageuously applied in the context of viruses such as human herpes viruses.

### Example 10

### Making and Using SPP Vectors According to the Present Invention

pSOLO™ HS3F is an example of the type of vector that can be constructed to achieve Kid-mediated Single Protein Production in living cells. Addition of Tetracycline to the growth medium induces PrTet. This simultaneously stimulates the synthesis of Kid and of any other gene cloned in the multicloning site (MCS) of the vector. Kid expression will stop cell growth without interfering with the ability of the host cell to transcribe/translate any gene lacking TTACT sites.

In this particular example, the gene of interest is tagged with 6His-Streptag and/or 3FLAG peptides. Also, TetR and Ampr lack TTACT sites.

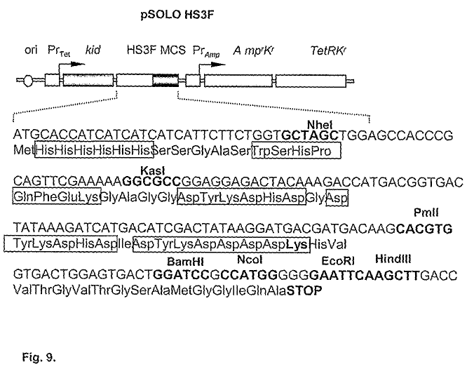

FIG. 9 shows detailed construction of pSOLO™ HS3F

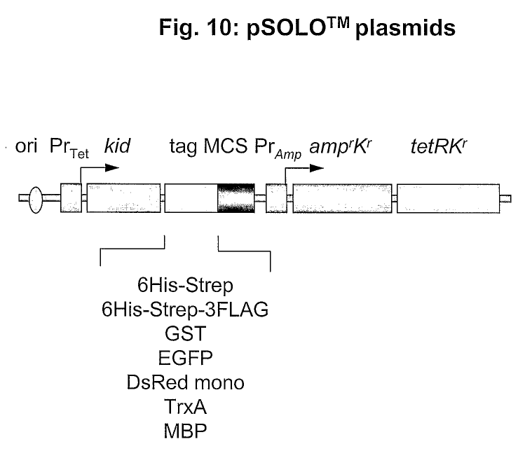

The same principles apply to the construction of other SPP vectors according to the present invention. For example, other exemplary pSOLO™ vectors are illustrated in FIG. 10.

These pSOLO™ vectors facilitate a novel approach for single protein production in E. coli using Kid toxin. This Kid-based single protein production (SPP) technology can be adapted to other existing technologies for protein expression/purification (e.g. kanr, chlr, GST, Thioredoxin, DsRed, EGFP and MBP, as examples).

Preferred examples are pSOLO-HS, pSOLO-HS3F, pSOLO-GST, pSOLO-EGFP, pSOLO-DsRed. Other examples include pSOLO-TrxA and pSOLO-MBP. A strength of the invention is that there is no restriction on the configuration of tags/purification moieties or other components of the gene of interest so long as TTACT sites can be removed.

The pSOLO™ vectors in this example are pSOLO™ plasmids. These advantageously permit expression of Kid and the selected protein when the promoter(s) is(are) induced. These also advantageously provide a low global protein synthesis background as the result of Kid activity. These vectors find application in single protein production (SPP) as described herein.

### Example 11

### Expression of Protein Using SPP Vectors

The invention provides for expression of Kid and the selected protein when the promoter(s) is(are) induced. In this example, Kid and various different proteins of interest are expressed from single promoters (bicistronic). It may be preferred to arrange Kid and the protein(s) of interest under the control of separate promoters (each being monocistronic).

In this example we demonstrate that Kid co-expression allows simultaneous expression of the gene of interest when said gene lacks TTACT sites. According to the present invention coding sequences lacking TTACT sites may be selected for expression, or more preferably the coding sequence of interest is mutated to remove TTACT sites.

Furthermore, the protein expression pattern of the pTET-expression vectors are compared with that of the pSOLO plasmids.

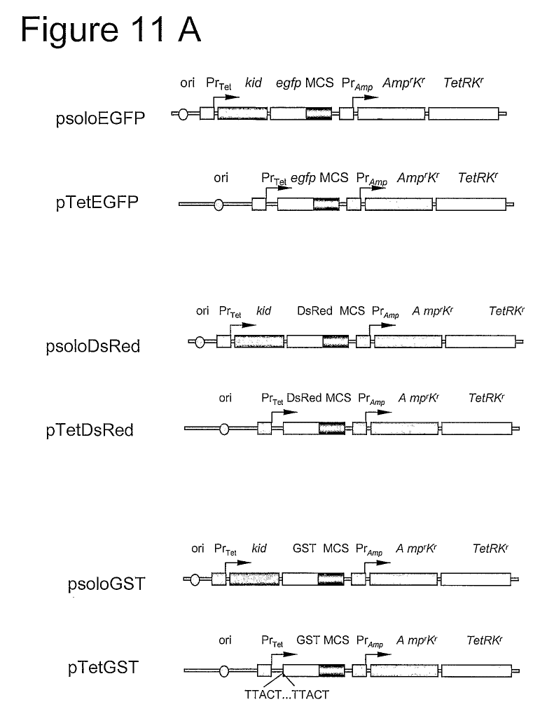

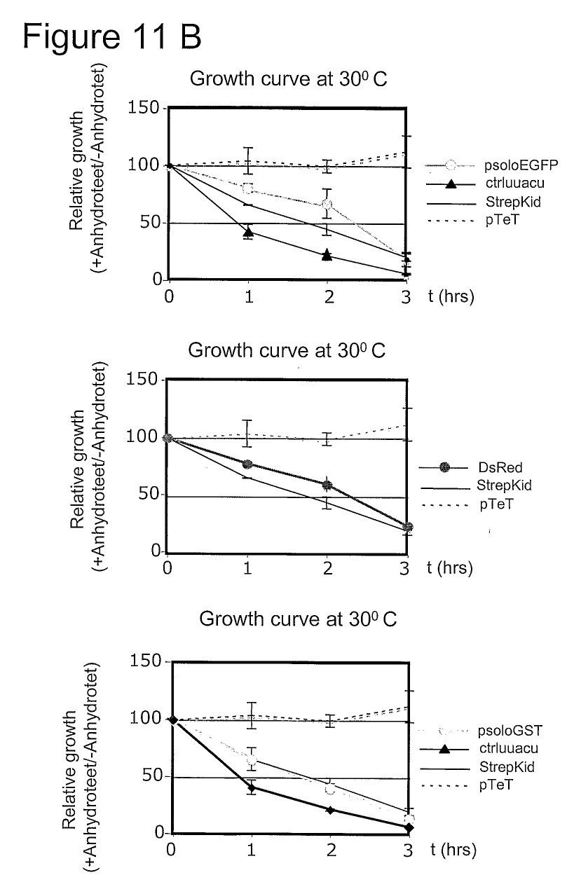

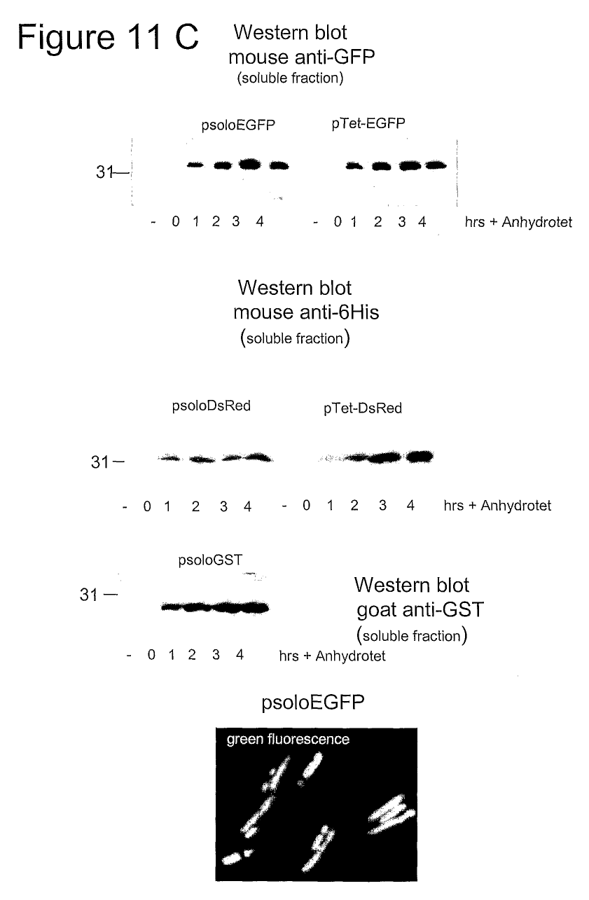

pSOLO™ plasmids bearing the gene of interest are constructed as above, as shown in FIG. 11A. These are introduced into host cells. Expression is then induced.

FIG. 11B shows that Kid inhibits cell growth of the pSOLO™ strains when the promoter is induced. FIG. 11C shows that the selected protein is expressed when the promoter is induced

The results are shown for example in FIG. 11C (gels are western-blots against the gene of interest.) Thus, it is shown that the expression of a gene of interest from pSOLO™ is as good as from the parental plasmid.

### Example 12

### Vectors and Methods for Sensitising Nucleotide Sequence to Kid

This example shows both expression and sensitisation of a gene of interest to Kid action according to the present invention.

The nucleic acid of interest is made sensitive to Kid/PemK endoribonuclease by a method comprising

(a) providing a nucleic acid;
(b) screening the nucleic acid for the nucleotide sequence UUACU or TTACT;
(c) mutating said sequence such that there is at least one occurrence of UUACU or TTACT.

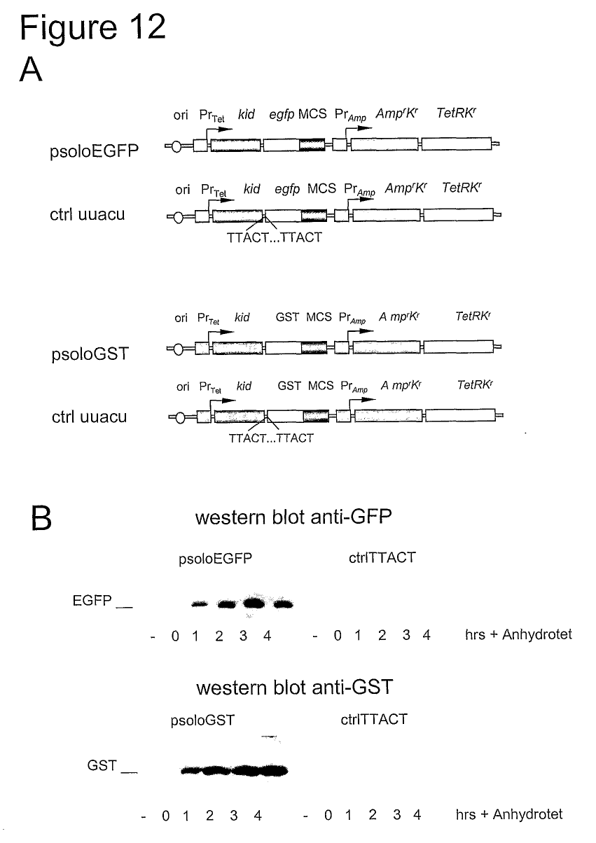

A vector is made by a method comprising selecting nucleic acid components for inclusion in said vector, mutating the sequence such that there is at least one occurrence of TTACT, and assembling the nucleic acid components to produce the vector. This is illustrated in FIG. 12A (‘ctrl uuacu’ in FIG. 12A).

In parallel, Kid resistant pSOLO™ vectors are made for the same polypeptide of interest (‘psolo’ in FIG. 12A).

FIG. 12B shows that Kid inhibits the synthesis of the selected protein when TTACT sites are introduced into the gene (‘ctrlTTACT’ lanes) and that protein production is excellent when those sites are absent (pSOLO™ vectors-‘psolo’ lanes).

### Example 13

### Optimisation—Protein Production with Low Background

It is an advantage of the invention that low global protein synthesis background is produced using the Kid/pSOLO™ system of single protein production (SPP).

We show here that the signal/background ratio to achieve SPP improves when using the pSOLO™ compared to its parental plasmids. These results demonstrate that the system works efficiently as described. Naturally conditions may be optmised according to the particular application to which the invention is put.

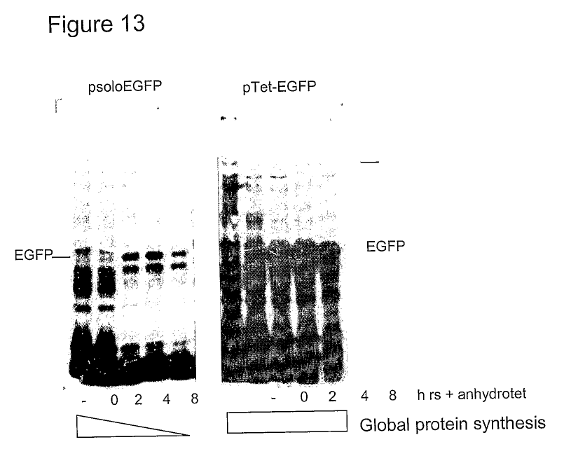

FIG. 13 shows 35S-Methionine in vivo labeling with the plasmids described in the previous example. Low protein synthesis background can be clearly observed; single protein production (SPP) according to the present invention is demonstrated.

### Example 14

### Optimisation—Protein Production at Different Temperatures

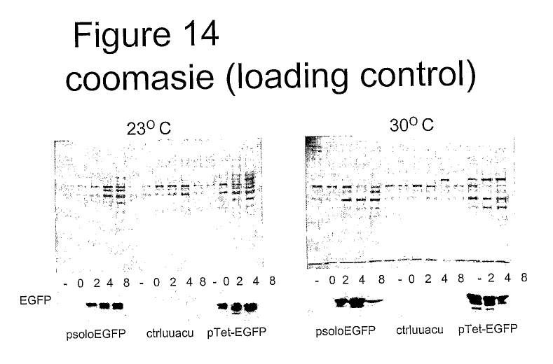

FIG. 14 shows Western blot anti-GFP showing improved signal/background ratio.

This alos shows protein expression at different temperatures (23° C., 30° C.). These results show that the pSOLO™ plasmids work well at low temperatures such as 23° C. as well as more standard temperatures such as 30° C.

### Example 15

### Further Optimisation of pSOLO™

This example illustrates improvement/optimisation of conditions for use of pSOLO™.

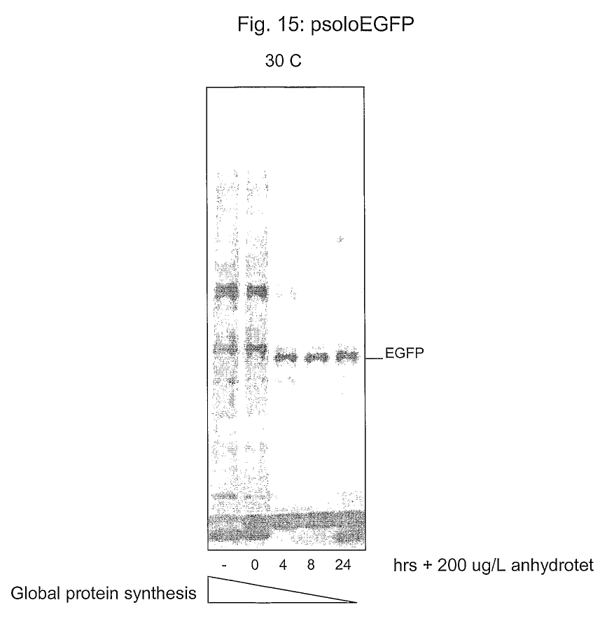

FIG. 15 shows both expression of EGFP after 24 hrs of induction and very low background signal.

In more detail, FIG. 15 shows improving signal/background ratio: 1 & 2; Testing protein expression at different temperatures (23° C./30° C.); and different Anhydrotetracycline concentrations (2 mg/L/200 ug/L).

All publications mentioned in the above specification are herein incorporated by reference. Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for engineering a nucleic acid for expression in the presence of Kid/PemK endoribonuclease comprising
(i) screening the nucleotide sequence of the nucleic acid for the sequence UUACU or TTACT
(ii) mutating said sequence such that there are no longer any occurrences of UUACU or TTACT.

2. A method according to claim 1 wherein each occurrence of UUACU or TTACT is mutated by replacing the first or last U/T of each occurrence.

3. A method according to claim 2 wherein said first or last U or T is mutated as a silent mutation.

4. A nucleic acid obtained by the method of claim 1.

5. A nucleic acid vector comprising an origin of replication and a nucleic acid according to claim 4.

6. A vector according to claim 5 wherein the origin or replication is a prokaryotic origin of replication capable of functioning in E. coli.

7-10. (canceled)

11. A method for making a vector comprising selecting nucleic acid components for inclusion in said vector, screening the nucleotide sequence of said components for the sequence TTACT, wherein if one or more TTACT sequences is found then said sequence is mutated such that there are no longer any occurrences of TTACT, and assembling the nucleic acid components to produce the vector.

12-14. (canceled)

15. A method of inhibiting cell growth comprising inducing RNA cleavage by Kid/PemK at the sequence UUACU in said cell.

16. A method of inducing apoptosis in a eukaryotic cell said method comprising causing cleavage of RNA at UUACU site(s) in said cell by contacting said cell with a composition that comprises Kid/PemK wherein said cleavage is mediated by Kid/PemK.

17. (canceled)

18. A method of making a ribonucleic acid resistant or sensitive to Kid/PemK endoribonuclease, said method comprising
(a) providing a nucleic acid;
(b) screening the nucleic acid for the nucleotide sequence UUACU or TTACT;
(c) mutating said sequence such that there are no longer any occurrences of UUACU or TTACT;
wherein when the nucleic acid of (a) is a deoxyribonucleic acid, said method further comprises
(d) transcribing said deoxyribonucleic acid to produce ribonucleic acid.

19-20. (canceled)

21. A method for making a vector comprising selecting nucleic acid components for inclusion in said vector, screening the nucleotide sequence of said components for the sequence TTACT, wherein if no TTACT sequence is found then said nucleotide sequence is mutated such that there is at least one occurrence of TTACT, and assembling the nucleic acid components to produce the vector.

22-31. (canceled)

32. A nucleic acid obtained by the method of claim 22.

33. A nucleic acid vector comprising an origin of replication and a nucleic acid according to claim 32.

34. A vector according to claim 33 wherein the origin or replication is a prokaryotic origin of replication capable of functioning in E. coli.


SEQUENCE LISTING (27 sequences)

SEQ ID NO: 1 - DNA, 31 bp, Artificial
  Oligonucleotide
ctaaagtaaagacttttctttgtggcgtagc

SEQ ID NO: 2 - DNA, 31 bp, Artificial
  Oligonucleotide
gctacgccacaaagaaaagtctttactttag

SEQ ID NO: 3 - DNA, 49 bp, Artificial
  Oligonucleotide
ggcgtagcatgctagatttctgatcgtttttggaattttgtggctggcc

SEQ ID NO: 4 - DNA, 49 bp, Artificial
  Oligonucleotide
ggccagccacaaaattccaaaaacgatcagaaatctagcatgctacgcc

SEQ ID NO: 5 - DNA, 26 bp, Artificial
  Oligonucleotide
cgcaggtcataagcagcagggaacgc

SEQ ID NO: 6 - DNA, 29 bp, Artificial
  Oligonucleotide
ggccgcgttccctgctgcttatgacctgc

SEQ ID NO: 7 - DNA, 21 bp, Artificial
  Oligonucleotide
agcaaaaccaccgacgctatc

SEQ ID NO: 8 - DNA, 21 bp, Artificial
  Oligonucleotide
ggttttcgttatccgcgcgac

SEQ ID NO: 9 - DNA, 22 bp, Artificial
  Oligonucleotide
taaatccacatcagaaccagtt

SEQ ID NO: 10 - DNA, 22 bp, Artificial
  Oligonucleotide
atgtcgcagagagaaaatgcag

SEQ ID NO: 11 - DNA, 21 bp, Artificial
  Oligonucleotide
cagcggccatttgtttctcag

SEQ ID NO: 12 - DNA, 24 bp, Artificial
  Oligonucleotide
gtgactgatcttcaccaaacgtat

SEQ ID NO: 13 - DNA, 22 bp, Artificial
  Oligonucleotide
gtttttcgcagaacttcagcgt

SEQ ID NO: 14 - DNA, 858 bp, Escherichia coli
gtgactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgggccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttcagtcccgtggaataaaacgtgcccgtgcgcgtcgtgatgcgaacagagaacgtcaggatatcgtcaccctagtgaaacggcagctgacgcgtgaaatctcggaaggacgcttcactgctaatggtgaggcggtaaaacgcgaagtggagcgtcgtgtgaaggagcgcatgattctgtcacgtaaccgcaattacagccggctggccacagcttctccctga

SEQ ID NO: 15 - DNA, 258 bp, Escherichia coli
atgcataccacccgactgaagagggttggcggctcagttatgctgaccgtcccaccggcactgctgaatgcgctgtctctgggcacagataatgaagttggcatggtcattgataatggccggctgattgttgagccgtacagacgcccgcaatattcactggctgagctactggcacagtgtgatccgaatgctgaaatatcagctgaagaacgagaatggctggatgcaccggcgactggtcaggaggaaatctga

SEQ ID NO: 16 - RNA, 104 nt, Escherichia coli
ugguagcagacgauuuuuacacccgcccacaccgucauaucuuuacugaaauggcgcguuugcaggaaagcgguagcccuaucgaucugauuacucuugcggaa

SEQ ID NO: 17 - RNA, 130 nt, Escherichia coli
auuaucggcauuauucguuacuacacccgugaggcgggcgugcguggucuggagcgugaaaucuccaaacugugucgcaaagcgguuaagcaguuacugcucgauaagucauuaaaacauaucgaaauua

SEQ ID NO: 18 - RNA, 319 nt, Escherichia coli
uauacuaaaguaaagacuuuacuuuguggcguagcaugcuagauuacugaucguuuaaggaauuuuguggcuggccacgccguaagguggcaaggaacugguucugauguggauuuacaggagccagaaaagcaaaaaccccgauaaucuucuucaacuuuggcgaauacgaaaagauuaccggggcccacuuaaaccguauagccaacaauucagcuaugcggggaguauaguuauaugcccggaaaaguucaagacuucuuucugugcucgcuccuucugcgcauuguaagugcaggauggugugacugaucuucac

SEQ ID NO: 19 - DNA, 858 bp, Escherichia coli; identical to SEQ ID NO: 14

SEQ ID NO: 20 - DNA, 333 bp, Escherichia coli
atggaaagaggggaaatctggcttgtctcgcttgatcctaccgcaggtcatgagcagcagggaacgcggccggtgctgattgtcacaccggcggcctttaatcgcgtgacccgcctgcctgttgttgtgcccgtaaccagcggaggcaattttgcccgcactgccggctttgcggtgtcgttggatggtgttggcatacgtaccacaggtgttgtacgttgcgatcaaccccggacaattgatatgaaagcacggggcggaaaacgactcgaacgggttccggagactatcatgaacgaagttcttggccgcctgtccactattctgacttga

SEQ ID NO: 21 - DNA, 720 bp, Artificial
  EGFP artificial sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 699 bp, Artificial
  Plasmid sequence GST-MCS from pGEX-2T
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgggaattcatcgtgactga

SEQ ID NO: 23 - DNA, 330 bp, Escherichia coli
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaa

SEQ ID NO: 24 - DNA, 1319 bp, Artificial
  Plasmid sequence malE-MCS from pMAL-X
atgaaaataaaaacaggtgcacgcatcctcgcattatccgcattaacgacgatgatgttttccgcctcaagcttcgcctatgacgttccggactacgcagctgcagaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatcctctagagtcgacctgcatggtaccgccagcttggcactggccgt

SEQ ID NO: 25 - DNA, 477 bp, Human papillomavirus type 18
atggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataa

SEQ ID NO: 26 - DNA, 191 bp, Artificial
  Partial pSOLO HS3F vector sequence
atgcaccatcatcatcatcattcttctggtgctagctggagccacccgcagttcgaaaaaggcgccggaggagactacaaagaccatgacggtgactataaagatcatgacatcgactataaggatgacgatgacaagcacgtggtgactggagtgactggatccgccatggggggaattcaagcttgacc

SEQ ID NO: 27 - protein, 62 aa, Artificial
  Partial pSOLO HS3F vector sequence
MHHHHHHSSGASWSHPQFEKGAGGDYKDHDGDYKDHDIDYKDDDDKHVVTGVTGSAMGGIQA